(19) United States
(12) Patent Application Publication
(10) Pub. No.: US 2024/0298969 A1
(43) Pub. Date: Sep. 12, 2024

(54) SYSTEMS AND METHODS FOR EXTENDED EGM COLLECTION AND UTILIZATION BY AN IMPLANTABLE MEDICAL DEVICE

(71) Applicant: Pacesetter, Inc., Sylmar, CA (US)

(72) Inventors: Jong Gill, Valencia, CA (US); Fujian Qu, San Jose, CA (US); Joanna Urbanski, Sylmar, CA (US); Simon Skup, Sylmar, CA (US)

(21) Appl. No.: 18/583,032

(22) Filed: Feb. 21, 2024

Related U.S. Application Data
(60) Provisional application No. 63/489,429, filed on Mar. 10, 2023.

(51) Int. Cl. A61B 5/00; A61B 5/0205; A61B 5/024; A61B 5/346

(52) U.S. Cl. CPC A61B 5/686; A61B 5/0006; A61B 5/0031; A61B 5/0205; A61B 5/7285; A61B 5/024; A61B 5/346

(57) ABSTRACT

An implantable medical device (IMD) includes one or more sensing circuits configured to sense one or more physiological characteristics and to generate physiological data indicative of the one or more physiological characteristics. An input is configured to receive a trigger. Responsive to receiving the trigger, a continuous data collection mode (CDCM) comprising a predetermined sampling rate is enabled. Physiological data is continuously generated. The physiological data is continuously stored in a buffer memory at the predetermined sampling rate for a duration of a collection session associated with the CDCM. The amount of data stored in the buffer memory during the collection session, including the physiological data, exceeds a capacity of the buffer memory. Connect and transmit operations are performed at a periodic communication interval during the collection session to connect with the external device and transmit at least a portion of the physiological data stored in the buffer memory.

### CROSS-REFERENCE TO RELATED APPLICATIONS

This application claims priority to U.S. Provisional Patent Application No. 63/489,429, filed 10Mar. 2023, entitled “SYSTEMS AND METHODS FOR EXTENDED EGM COLLECTION AND UTILIZATION BY AN IMPLANTABLE MEDICAL DEVICE”, the subject matter of which is incorporated herein by reference in its entirety.

### FIELD OF THE INVENTION

Embodiments herein generally relate to implantable medical devices, and more particularly to improving extended data capture by the implantable medical device based on a triggering event.

### BACKGROUND

An implantable medical device (“IMD”) is a medical device that is configured to be implanted within a patient anatomy and commonly employ one or more electrodes that either receive or deliver voltage, current or other electromagnetic pulses (generally “energy”) from or to an organ or tissue for diagnostic or therapeutic purposes. In general, IMDs include a battery and electronic circuitry. The electronic circuity, such as a pulse generator, and/or a microprocessor, is configured to handle RF communication with an external device. Additionally or alternatively, the electronic circuitry is configured to control patient therapy.

IMDs are programmed by, and transmit data to, external devices controlled by physicians and/or the patient. The external devices communicate through wireless communication links with the IMDs. For example, the IMDs can communicate with the external devices using commercial protocols, such as the Bluetooth Low Energy (BLE) protocol and other protocols which are compatible with commercial wireless devices such as tablet computers, smartphones, and the like. By enabling a commercial wireless device to communicate with the IMD using a commercial protocol, the physician and/or patient may easily and/or frequently activate communication between the IMD and the external device.

Certain conventional IMDs continuously track intracardiac electrogram (e.g., IEGM, EGM) data in order to track many of the vital cardiac functions and status such as heart rate, arrhythmia, atrio-ventricular and ventricular-ventricular intervals, etc. Even though the IMD continuously tracks the EGM for monitoring, the actual amount of EGM episodes saved in the IMD memory is relatively small, limited by the memory space available. The growing capacity in computation and sophisticated approach using artificial intelligence (Al) and/or machine learning for improved diagnostics and predictive analytics requires a longer duration of continuous data that cannot be satisfied by the limited memory space in the IMD.

A need exists for improved methods and systems for capturing and providing continuously acquired data to improve disease detection and management.

### SUMMARY

In accordance with embodiments herein, an implantable medical device (IMD), comprises one or more sensing circuits, an input, a transceiver circuit, a memory, and one or more processors. The one or more sensing circuits are configured to sense one or more physiological characteristics and to generate physiological data indicative of the one or more physiological characteristics. The input is configured to receive a trigger, and the transceiver circuit is configured to communicate with an external device. The memory is configured to store program instructions and comprises a buffer memory. The one or more processors, when executing the program instructions, are configured to: responsive to receiving the trigger: enable a continuous data collection mode (CDCM) comprising a predetermined sampling rate; continuously generate the physiological data; continuously store the physiological data in the buffer memory at the predetermined sampling rate for a duration of a collection session associated with the CDCM, the amount of data stored in the buffer memory during the collection session, including the physiological data, exceeding a capacity of the buffer memory; connect with the external device; and transmit at least a portion of the physiological data stored in the buffer memory to the external device, wherein the connect and transmit operations are performed at a periodic communication interval during the collection session.

Optionally, the CDCM is further configured to store the physiological data at the predetermined sampling rate for a predetermined duration. Optionally, the periodic communication interval is determined based on i) a capacity of the buffer memory, ii) a number of sensing channels configured to sense cardiac activity (CA) signals during the collection session, iii) the predetermined sampling rate, iv) a capacity of a memory space of the external device, v) data transfer speed between the IMD and the external device, and/or vi) time to establish connection between the IMD and the external device.

Optionally, the physiological data includes i) heart sounds, ii) blood glucose data, iii) pulse oximetry, iv) CA signals, v) temperature, vi) heart rate, vii) impedance, viii) blood pressure, ix) blood oxygen saturation, x) activity, xi) posture, xii) nerve activity, xiii) blood sugar level, or xiv) cholesterol level. Optionally, the trigger is i) a communication from the external device, ii) a physiological trigger from a physiological sensor located within the IMD or external to the IMD, or iii) generated in response to a physiological condition. Optionally, wherein the one or more processors is further configured to disable the CDCM based on i) a predetermined end time, ii) a predetermined duration, iii) a predetermined number of transmissions, or iv) receipt of a disabling message from a sensor or the external device.

Optionally, wherein the one or more processors is further configured to determine a physiological feature, wherein in response to the physiological feature exceeding a threshold, the one or more processors are further configured to disable the CDCM. Optionally, the physiological feature is a heart rate. Optionally, wherein, in response to receiving a disabling message, the one or more processors is further configured to disable the CDCM. Optionally, wherein the connect operation further comprises connecting with the external device at least a first time and a second time, wherein the transmit operation further comprises transmitting a first set of data during the first time and transmitting a second set of data during the second time that is different from the first set of data.

In accordance with embodiments herein, a computer implemented method comprises, responsive to receiving, by an implantable medical device (IMD), a trigger enabling a continuous data collection mode (CDCM) on the IMD, the CDCM having an associated duration of a collection session. The method further comprises continuously sensing physiological characteristics, and storing, at a predetermined sampling rate associated with the CDCM, physiological data associated with the sensed physiological characteristics in a buffer memory within the IMD, wherein an amount of data, including the physiological data, to be stored in the buffer memory during the collection session exceeds a capacity of the buffer memory. The method further comprises transmitting the data stored in the buffer memory from the IMD to an external device at a periodic communication interval set to prevent the data in the buffer memory from being overwritten during the collection session.

Optionally, the method further comprises identifying one or more sensing channel associated with the CDCM, the one or more sensing channel included within the IMD; wherein the continuously sensing further comprises continuously sensing physiological characteristics using the one or more sensing channel, wherein the physiological characteristics comprises cardiac activity (CA) signals; and storing, at the predetermined sampling rate, the data associated with the physiological characteristics sensed on the one or more sensing channel in the buffer memory.

Optionally, the transmitting of the method further comprises transmitting a first set of data to the external device at a first time based on the periodic communication interval; and transmitting a second set of data to the external device at a second time based on the periodic communication interval, wherein the second time is subsequent to the first time, wherein the second set of data was sensed subsequently with respect to the first set of data.

Optionally, the method further comprises combining the first set of data and the second set of data temporally. Optionally, the method further comprises determining a treatment based on a combined dataset including the first set of data and the second set of data.

Optionally, in response to enabling the CDCM, the method further comprises identifying marker data, wherein the marker data includes i) timing of QRS, ii) arrhythmia detection and termination, iii) timing of sensing, iv) noise, v) activity, vi) sleep, vii) physiological events, or viii) posture of patient, wherein the physiological data is EGM data, and the storing further comprises storing the marker data with the EGM data, wherein the marker data is temporally associated with the EGM data.

Optionally, the periodic communication interval is determined based on i) a capacity of the buffer memory, ii) a number of sensing channels associated with the CDCM, or iii) the predetermined sampling rate. Optionally, the method further comprises determining a physiological feature; and in response to the physiological feature exceeding a threshold, disabling the CDCM. Optionally, the method further comprises, responsive to receiving, by the IMD, a second trigger, disabling the CDCM on the IMD. Optionally, the method further comprises, responsive to the CDMC being disabled, transmitting the data stored in the buffer memory from the IMD to the external device.

### BRIEF DESCRIPTION OF THE DRAWINGS

FIG. 1 illustrates a simplified diagram of a system for continuous data collection using an implantable medical device (IMD) and transfer of the continuously acquired data at a periodic communication interval to an external device in accordance with embodiments herein.

FIG. 2 illustrates a block diagram of an exemplary IMD that is configured to be implanted into the patient in accordance with embodiments herein.

FIG. 3 illustrates another block diagram of internal components of an exemplary IMD in accordance with embodiments herein.

FIG. 4 illustrates a flowchart of a method for programming continuous data collection mode(s) (CDCM(s)) for an IMD in accordance with embodiments herein.

FIG. 5 illustrates a flowchart of a method from the perspective of the IMD for enabling the CDCM and acquiring continuous physiological data in accordance with embodiments herein.

FIG. 6 illustrates a flowchart of a method from the perspective of the external device for enabling the CDCM on the IMD and initiating the acquisition of continuously acquired data in accordance with embodiments herein.

FIG. 7 illustrates a flowchart of a method for managing the storing of the continuously acquired data by the IMD and transferring of the continuously acquired data between the IMD and the external device in accordance with embodiments herein.

FIG. 8 is a graphical depiction of several data units of continuously acquired data that are assigned consecutive Index numbers in accordance with embodiments herein.

FIG. 9 illustrates a flowchart of a method for disease diagnostics and determining physiological-based features for biomarker estimate utilizing the continuously acquired data in accordance with embodiments herein.

FIG. 10 illustrates the distributed processing system in accordance with embodiments herein.

FIG. 11 illustrates a functional block diagram of the external device that is operated in accordance with the processes described herein and to interface with the IMD and/or physiological sensor as described herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

### Terms

The terms “continuous data collection mode” and “CDCM” shall mean a mode during which an IMD continuously samples data, such as CA data, EGM data and/or other types of physiological data measured by the IMD, other sensors, and/or sensing circuits, that has been sensed from a patient without a break in time. Cardiac activity (CA) signals can be sensed by electrodes within the patient's body over one, two, or more channels. The signals can be stored in a memory space at one or more predetermined sampling rate(s) (e.g., different channels can be sampled at the same or different rates). Other physiologic data can also be sensed by one or more sensing circuits, over at least one sensing channel associated with the sensing circuit, and stored in the memory space. The CDCM can be directly enabled/disabled upon receipt of a trigger, such as through telemetry (e.g., a message from an external device, implanted sensor, external sensor to the patient and/or IMD), and/or enabled/disabled automatically by preset parameters (e.g., time of the day, a diagnostic parameter exceeding a threshold, the length of time CDCM has been active), an enabling message or disabling message from a sensor/device contained within the IMD or outside the IMD, and the like. In some embodiments, other detected and/or programmed conditions can trigger the CDCM to be enabled or disabled. The CDCM can be programmed to continuously acquire data for a predetermined collection session, a collection session occurring within a predetermined time period, be disabled manually and/or when a parameter exceeds a threshold, etc. Each CDCM can also be customized to different sampling frequencies, different sensing channels, different durations, and different triggers, etc.

The terms “enable” and “enabled” shall mean turning on and/or activating a CDCM.

The terms “disable” and “disabled” shall mean turning off and/or deactivating a CDCM.

The term “continuously acquired data”, “continuous acquired data”, “continuously sensed data”, and “continuous sensed data” are used interchangeably herein and shall mean data that is sampled at a specified sampling frequency without a break in time. The continuously acquired data can be continually sensed at the specified sampling frequency that can be equal or lower than the sample rate at which sensing circuitry and/or sensing channel(s) sense raw CA data. One or more of the types of data can be continuously acquired, temporally correlated, and stored. In some cases, different types of continuously acquired data are stored in the same dataset. The continuously acquired data can include one or more of CA, EGM, heart sound, blood glucose data, pulmonary artery pressure, pulse oximetry, etc., as well as marker data.

The term “marker data” shall mean device-generated annotation data corresponding to device-determined events in the continuously acquired data, such as timing of QRS, timing of sensing, arrhythmia detection and termination, noise, activity, sleep, physiological events, posture of patient, etc.

The term “trigger” shall mean an indicator that indicates to the IMD to enable or disable the CDCM. The trigger can be an activation or deactivation signal from an external device, from within the IMD, from a sensor, be generated in response to a physiological feature or parameter exceeding a threshold, be generated based on a specific time, a specific time period, on a periodic basis, and the like.

The term “periodic communication interval” shall mean the amount of time between consecutive telemetry connections between the IMD and an external device, wherein the connection is established to transmit the continuously acquired data to the external device. The periodic communication interval can be based on one or more of i) the size or capacity of the memory space storing the continuously acquired data, ii) the number of channels used to acquire the data, iii) the sampling rate, iv) an expected connection time needed to establish the connection between the IMD and the external device, v) the size or capacity of the memory space of the external device receiving the continuously acquired data, and vi) a data transfer speed between the IMD and the external device. The periodic communication interval is determined such that the continuously acquired data is not overwritten in the memory before the data can be transferred to the external device. Advantageously, the periodic communication interval can be determined to maximize the amount of time between establishing the consecutive telemetry connections, thus conserving the battery power of the IMD as well as ensuring no loss of the continuously acquired data.

The term “collection session” shall mean a length of time during which CA, EGM, sensor, and/or other data is acquired and/or continuously acquired. The length of the collection session can be predetermined by the CDCM, can be predetermined to occur within a predetermined time period (e.g., specific time of day, day of week), and/or can continue in time until disabled manually, upon receipt of a trigger, when a parameter exceeds a threshold, and/or when a maximum duration in time has been reached.

The term “data unit” shall mean portions of continuously acquired data at a sampling rate, and in some cases associated marker data, that are stored in a memory and indexed. Each data unit includes data at a specific point in time, and can include CA signals, such as one or more EGM samples, depending upon the number of selected channels.

The term “last read index” shall mean the last data unit that was read by an external device.

The term “maximum index” shall mean a maximum number of data units that can be acquired during a current CDCM session. The maximum index prevents the continued use of device resources (e.g., battery depletion) of the IMD in the event that the CDCM is not disabled by the IMD, the external device, or other trigger.

The term “posture” shall mean postural states and/or activity levels of a patient including supine, prone, lying on a right side, lying on a left side, standing (upright), and the like. The term “upright posture” shall mean any and all postures of the patient when the patient is in upright posture including standing or sitting. The upright posture may be identified by an accelerometer reading based on accelerometer data detected and associated with various postures.

The term “acceleration signature” shall mean signals detected by an accelerometer or other sensor associated with or of the IMD that are indicative of heart sounds generated during cardiac beats. The acceleration signatures can be analyzed for activity level and can be indicative of heart sounds generated in connection with different postures of a patient.

The terms “physiologic data” and “physiological data” shall mean one or more of physiologic conditions or traits of the patient. For example, the physiologic data may represent cardiac activity signals (CA signals) sensed by electrodes positioned within or about the heart. The CA signals may also be sensed by electrodes provided on the housing of the IMD. As another example, the physiologic data may represent impedance signals, respiratory signals, heart sounds, heart rate, temperature, blood glucose data, pulmonary artery pressure, blood pressure, pulse oximetry, nerve activity (e.g., as measured within the spinal column or dorsal root), brainwave activity and the like. The physiologic data may represent pulse oximetry signals, blood oxygen saturation, cholesterol level, blood sugar levels, activity of the patient, posture of the patient, and the like. For example, one or more sensing circuits can sense one or more physiological characteristics and generate physiological data indicative of the one or more physiological characteristics.

The term “subcutaneous” shall mean below the skin, but not intravenous. For example, a subcutaneous electrode/lead does not include an electrode/lead located in a chamber of the heart, in a vein on the heart, or in the lateral or posterior branches of the coronary sinus.

The terms “processor,” “a processor”, “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “external device” shall mean a commercial wireless device (e.g., a tablet computer, a smartphone, a laptop computer) and/or a specialized wireless device such as a programmer or bedside monitor. A patient, using an application, button, selection, etc., on the external device, may trigger the external device to transmit signals from the external device to the IMD. The transmitted signals include a connection request that the IMD establish a communications link with the external device. The application may be written to be compatible with numerous operating systems. When the connection request is detected by the IMD, the IMD enters a communication initialization mode and implements a pairing and/or bonding procedure. The pairing and/or bonding procedure may be performed based on various wireless protocols (e.g., Bluetooth Low Energy (BLE), Bluetooth, ZigBee). The pairing and/or bonding procedure may include various levels of complexity and security. For example, the procedure may include added security such as exchanging information to generate passkeys in both the IMD and the external device to establish a secure bi-directional communication link.

The terms “cardiac signals”, “cardiac activity”, “cardiac activity signal”, “cardiac activity signals”, “CA signal” and “CA signals” (collectively “CA signals”) are used interchangeably throughout and shall mean measured signals indicative of cardiac activity by a region or chamber of interest. For example, the CA signals may be indicative of impedance, electrical or mechanical activity by one or more chambers (e.g., left or right ventricle, left or right atrium) of the heart and/or by a local region within the heart (e.g., AV node, along the septal wall, within the left or right bundle branch, within the Purkinje fibers). The cardiac activity may be normal/healthy or abnormal/proarrhythmic. An example of CA signals includes EGM/IEGM signals. Electrical based CA signals refer to an analog or digital electrical signal recorded by two or more electrodes, where the electrical signals are indicative of cardiac activity. Heart sound (HS) based CA signals refer to signals output by a heart sound sensor such as an accelerometer, where the HS based CA signals are indicative of one or more of the S1, S2, S3 and/or S4 heart sounds. Impedance based CA signals refer to impedance measurements recorded along an impedance vector between two or more electrodes, where the impedance measurements are indicative of cardiac activity.

The term “treatment notification” shall mean a communication and/or device command to be conveyed to one or more individuals and/or one or more other electronic devices, including but not limited to, network servers, workstations, laptop computers, tablet devices, smart phones, IMDs, external diagnostic test (EDT) equipment and the like. When a treatment notification is provided as a communication, the treatment notification may present in an audio, video, vibratory or other user perceivable medium. The communication may be presented in various formats, such as to display patient information, messages, user directions and the like. The communication is presented on one or more of the various types of electronic devices described herein and may be directed to a patient, a physician, various medical personnel, various patient record management personnel and the like. The communication may represent an identification of a patient diagnosis and various treatment recommendations. The diagnosis and treatment recommendation may be provided directly to the patient. For example, in some circumstances, a diagnosis and treatment recommendation may be to modify a dosage level, in which case, the notification may be provided to the physician or medical practitioner. As another example, the diagnosis and treatment recommendation may be to begin, change or end certain physical activities, in which case, the notification may be provided to the patient, in addition to the physician or medical practitioner. As another example, the treatment notification may present an indication that a patient may or may not be a good candidate suited for implant of a ventricular assist device (e.g., LV assist device), a transplant, a valve repair procedure (e.g., a MitraClip™ valve repair to correct mitral regurgitation) and the like. Other nonlimiting examples of a communication type notification include, in part or in whole, a recommendation to schedule an appointment with a physician, schedule an appointment for additional blood work, perform an additional at home POC blood analysis (e.g., utilizing at home EDT equipment), recommend that the patient collect additional EDT and/or IMD data. When a notification includes an action that may be performed by a patient alone, the notification may be communicated directly to the patient. Other nonlimiting examples of a communication type notification include communications sent to a patient (e.g., via a PDE device or other electronic device), where the communication informs the patient of how a patient's lifestyle choices are directly affecting the patient's health. For example, when a patient consumes too much sugar, a notification may be sent to the patient to inform them that the excessive sugar has caused a spike in the patient's glucose level. As another example, when a patient avoids exercise for a period of time, the notification may inform a patient that the patient's lack of exercise has raised a PAP trend and/or introduced an undue burden on a patient's kidneys.

When a treatment notification is provided as a device command, the treatment notification may represent an electronic command directing a computing device (e.g., IMD, EDT equipment, local external device, server) to perform an action. For example, the action may include directing the following:
1. IMD or EDT equipment to provide additional IMD data and/or EDT data already available;
2. IMD or EDT equipment to collect additional data and/or another type of data;
3. IMD to deliver a therapy and/or modify a prior therapy (e.g., a pacing therapy, neural stimulation therapy, appetite suppression therapy, drug delivery rate);
4. Local external device to provide additional information regarding past and present behavior of the patient; and
5. Server to analyze further information in the patient medical record and/or from another medical record.

The term “treatment recommendation” shall mean a recommendation for the patient, medical personnel and/or a device (e.g., an IMD, local external device, remote server, or body generated analyte (BGA) device) to take an action and/or maintain a current course of action. Non-limiting examples of treatment recommendations include dispatching an ambulance to the patient's location, instructing the patient immediately go to a hospital, instructing the patient schedule an appointment, instructing the patient change a prescription, instructing the patient undergo additional examinations (e.g., diagnostic imaging examinations, exploratory surgery and the like), instructing the patient undergo a POC test to collect new BGA data, instructing the patient take a nutritional supplement (e.g., an ONS), instructing the patient start, stop or change a physical activity, or instructing the patient make no changes. The treatment recommendation may include an instruction to change, maintain, add or stop a therapy delivered by an active IMD, such as a pacing therapy, an ATP pacing therapy, a neural stimulation therapy, mechanical circulatory support, and the like.

The term “health care system” shall mean a system that includes equipment for measuring health parameters, and communication pathways from the equipment to secondary devices. The secondary devices may be at the same location as the equipment, or remote from the equipment at a different location. The communication pathways may be wired, wireless, over the air, cellular, in the cloud, etc. In one example, the healthcare system provided may be one of the systems described in U.S. published application US20210020294A1, entitled “METHODS DEVICE AND SYSTEMS FOR HOLISTIC INTEGRATED HEALTHCARE PATIENT MANAGEMENT” filed Jul. 16, 2020, the entire contents of which are incorporated in full by reference herein. Other patents that describe example monitoring systems include U.S. Pat. No. 6,572,557 entitled “SYSTEM AND METHOD FOR MONITORING PROGRESSION OF CARDIAC DISEASE STATE USING PHYSIOLOGIC SENSORS”, filed Dec. 21, 2000; U.S. Pat. No. 6,480,733 entitled “METHOD FOR MONITORING HEART FAILURE”, filed Dec. 17, 1999; U.S. Pat. No. 7,272,443 entitled “SYSTEM AND METHOD FOR PREDICTING A HEART CONDITION BASED ON IMPEDANCE VALUES USING AN IMPLANTABLE MEDICAL DEVICE”, filed Dec. 14, 2004; U.S. Pat. No. 7,308,309 entitled “DIAGNOSING CARDIAC HEALTH UTILIZING PARAMETER TREND ANALYSIS”, filed Jan. 11, 2005; and U.S. Pat. No. 6,645,153 entitled “SYSTEM AND METHOD FOR EVALUATING RISK OF MORTALITY DUE TO CONGESTIVE HEART FAILURE USING PHYSIOLOGIC SENSORS”, filed Feb. 7, 2002, the entire contents of which are incorporated in full by reference herein.

The term “IMD” shall mean an implantable medical device. Embodiments may be implemented in connection with one or more implantable medical devices (IMDs). Non-limiting examples of IMDs include one or more of neurostimulator devices, implantable leadless monitoring and/or therapy devices, and/or alternative implantable medical devices. For example, the IMD may represent a subcutaneous cardioverter defibrillator, cardiac monitoring device, pacemaker, cardioverter, cardiac rhythm management device, defibrillator, neurostimulator, leadless monitoring device, leadless pacemaker, left atrial or pulmonary artery pressure sensor, blood glucose monitoring device, and the like. The IMD may measure electrical, mechanical, impedance, blood glucose, or pressure information. For example, the IMD may include one or more structural and/or functional aspects of the device(s) described in U.S. Pat. No. 9,333,351, entitled “Neurostimulation Method And System To Treat Apnea” issued May 10, 2016 and U.S. Pat. No. 9,044,610, entitled “System And Methods For Providing A Distributed Virtual Stimulation Cathode For Use With An Implantable Neurostimulation System” issued Jun. 2, 2015, and U.S. patent application Ser. No. 17/820,654, entitled “System and Method for Intra-Body Communication of Sensed Physiologic Data”, filed Aug. 18, 2022, which are hereby incorporated by reference herein in their entireties. The IMD may monitor transthoracic impedance, such as implemented by the CorVue algorithm offered by St. Jude Medical. Additionally or alternatively, the IMD may include one or more structural and/or functional aspects of the device(s) described in U.S. Pat. No. 9,216,285, entitled “Leadless Implantable Medical Device Having Removable And Fixed Components” issued Dec. 22, 2015 and U.S. Pat. No. 8,831,747, entitled “Leadless Neurostimulation Device And Method Including The Same” issued Sep. 9, 2014, which are hereby incorporated in full by reference herein. Additionally or alternatively, the IMD may include one or more structural and/or functional aspects of the device(s) described in U.S. Pat. No. 8,391,980, entitled “Method And System For Identifying A Potential Lead Failure In An Implantable Medical Device” issued Mar. 5, 2013 and U.S. Pat. No. 9,232,485, entitled “System And Method For Selectively Communicating With An Implantable Medical Device” issued Jan. 5, 2016, which are hereby incorporated in full by reference herein. Additionally or alternatively, the IMD may be a subcutaneous IMD that includes one or more structural and/or functional aspects of the device(s) described in U.S. Pat. No. 10,765,860, entitled “Subcutaneous Implantation Medical Device With Multiple Parasternal-Anterior Electrodes” issued Sep. 8, 2020; U.S. Pat. No. 10,722,704, entitled “Implantable Medical Systems And Methods Including Pulse Generators And Leads” issued Jul. 28, 2020; U.S. Pat. No. 11,045,643, entitled “Single Site Implantation Methods For Medical Devices Having Multiple Leads”, issued Jun. 29, 2021; and U.S. published application US20210330239A1, entitled “Method and system for adaptive-sensing of electrical cardiac signals” filed Mar. 4, 2021, which are hereby incorporated by reference herein in their entireties. Further, one or more combinations of IMDs may be utilized from the above incorporated patents and applications in accordance with embodiments herein. Embodiments may be implemented in connection with one or more subcutaneous implantable medical devices (S-IMDs). For example, the S-IMD may include one or more structural and/or functional aspects of the device(s) described in U.S. Pat. No. 10,722,704, entitled “IMPLANTABLE MEDICAL SYSTEMS AND METHODS INCLUDING PULSE GENERATORS AND LEADS”, issued Jul. 28, 2020 and U.S. Pat. No. 10,765,860, entitled “SUBCUTANEOUS IMPLANTATION MEDICAL DEVICE WITH MULTIPLE PARASTERNAL-ANTERIOR ELECTRODES”, issued Sep. 8, 2020, which are hereby incorporated by reference in their entireties. The IMD may represent a passive device that utilizes an external power source, an entirely mechanical plan will device, and/or an active device that includes an internal power source. The IMD may deliver some type of therapy/treatment, provide mechanical circulatory support, and/or merely monitor one or more physiologic characteristics of interest (e.g., PAP, CA signals, impedance, heart sounds).

Additionally or alternatively, embodiments herein may be implemented in connection with the methods and systems described in U.S. Published Application US20210350931A1, entitled “METHOD AND SYSTEMS FOR HEART CONDITION DETECTION USING AN ACCELEROMETER” filed Mar. 8, 2021, which is incorporated by reference herein in its entirety.

Additionally or alternatively, embodiments herein may be implemented in connection with the methods and systems described in U.S. Pat. No. 10,517,134, entitled “Method and system for managing communication between external and implantable devices” issued Dec. 24, 2019, U.S. Pat. No. 10,582,444, entitled “Implantable medical device with secure connection to an external instrument” issued Mar. 3, 2020, U.S. Pat. No. 9,889,305, entitled “Systems and methods for patient activated capture of transient data by an implantable medical device” issued Feb. 13, 2018, and U.S. Published application 20130204147, entitled “Atrial Fibrillation Detection Based On Pulmonary Artery Pressure Data”, filed Feb. 3, 2012, which are incorporated by reference herein in their entirety.

### System Overview

Various embodiments described herein include a method and/or system for managing scheduled, device/sensor-activated, and/or patient-activated capture of continuously acquired data by an implantable medical device (IMD) and/or sensors/sensing circuits and the transfer of the continuously acquired data to an external device using a communication link between the external device and the IMD. Once a continuous data collection mode (CDCM) is initiated, the IMD and/or sensors/sensing circuits record the continuously acquired data in data units (e.g., portions of continuous data) in a buffer memory. The data units are identified using an indexing method. The communication link is periodically established between the IMD (and/or sensors/sensing circuits) and the external device, such as at the periodic communication interval, to ensure that the continuously acquired data is transferred to the external device before the buffer memory is full and continuously acquired data is overwritten. But for the improvements described herein, a conventional IMD would record over continuously acquired data that the physician may otherwise want to review. The indexing method facilitates the tracking of the continuously acquired data to identify whether any data units are missing, and ensures that data units that were transmitted during different communication sessions are properly combined.

The transferred continuously acquired data may be physiologic data of interest that occurred over a period of time. The physiologic data of interest may be one or more of EGM data, CA signals, impedance signals, respiratory signals, heart sounds, heart rate, temperature, blood glucose data, blood pressure including but not limited to pulmonary artery pressure, pulse oximetry, nerve activity, brainwave activity, pulse oximetry signals, blood oxygen saturation, cholesterol related information, blood sugar levels, activity of the patient, posture of the patient, and the like. For example, the continuously acquired data can be EGM data and various marker data such as timing of QRS, arrhythmia detection and termination, noise, activity, sleep, physiological events, posture of patient, etc., that occurred temporally with the EGM data. In other embodiments, physiologic data can be continuously acquired without also acquiring the EGM data. The transferred physiologic data can be further transferred, as needed, to another system storage location so that the physiologic data may be available to a physician for review. The transferred physiologic data will allow the physician to review physiologic data corresponding to the scheduled, patient-initiated, and/or device-initiated CDCM to understand the health of the patient.

The patient can select a CDCM based on symptoms, correlated events such as other procedures such as dialysis, cardiac stress test, active symptoms, activities such as running, sleeping, or resting, etc. In other embodiments, the CDCM can be enabled automatically, such as based on a time of day, a predetermined time period associated with one or more of dates, times, etc., in response to a trigger from a sensor, in response to a diagnostic parameter exceeding a threshold, and the like.

The CDCMs can thus be enabled and disabled, and the continuously acquired data collected, without the patient and medical practitioner interfacing in person or through telemedicine. Some CDCMs require no action on the part of the patient, except for keeping their external device in proximity to their person. The medical practitioner can access the continuously acquired data from any eligible device, providing opportunities for remote review, collaboration, collection of many datasets over time, comparison of datasets of different patients, temporal correlation of the continuously acquired data with other patient data (e.g., data collected by other sensor(s)), and the like.

A technical effect of the various embodiments herein is to facilitate recording, in a buffer memory, continuously acquired data (e.g., EGM data, marker data, physiologic data) at a specified sampling rate and periodically transferring the continuously acquired data to an external device while continuing to continuously acquire data until the CDCM is disabled. A technical effect of recording the continuously acquired data is that the physician is provided with longer continuously acquired datasets that are ideal for identifying clinically meaningful physiological status that EGMs and/or other physiological data are representative of. For example, significant morphology alternations are present during hypoglycemia, and continuous transmission of the EGM during a suspected period of hypoglycemia will provide a meaningful signal averaged T wave morphology instead of few snippets of T wave morphology changes that may be transient in nature. A technical effect of various embodiments herein allows full performance validation of newly developed algorithms using the continuous EGM and/or other physiological data collection. A technical effect of various embodiments herein utilizes CDCM as to timing of activation, duration of data capture, mode of activation, additional data collected, treatment notification, and/or treatment recommendation, to further improve the performance of disease treatment, disease detection, and monitoring.

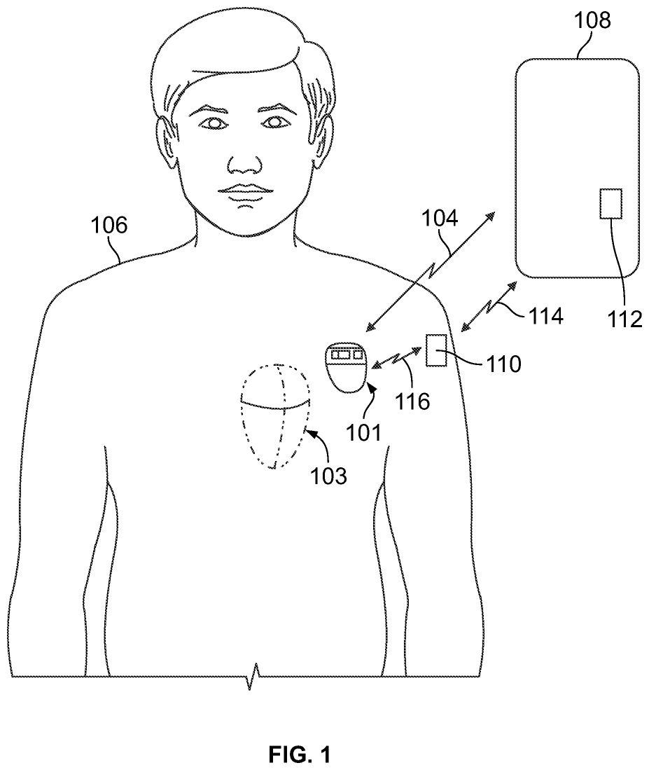

FIG. 1 illustrates a simplified diagram of a system for continuous data collection using an implantable medical device (IMD) 101 and transfer of the continuously acquired data at a periodic communication interval to an external device 108 in accordance with embodiments herein. The external device 108 may represent a tablet computer, smartphone, laptop, programmer, or the like. The external device 108 may program the IMD 101 and/or receive data from the IMD 101 via a communication link 104, such as by using an application (“App”) 112. For example, the external device 108 may transmit a request to the IMD 101. The request is received by the IMD 101, and in response to the request, the IMD 101 may enable or disable a CDCM, transfer continuously acquired data from a buffer memory section of the IMD 101 to the external device 108, as well as other processes described herein. The communication link 104 may use any standard wireless protocol such as a Bluetooth Low Energy (BLE) protocol, Bluetooth protocol, Wireless USB protocol, Medical Implant Communication Service (MISC) protocol, ZigBee protocol, and/or the like that define a means for transmitting and receiving information (e.g., data, commands, instructions) between devices.

The IMD 101 may be implanted within a patient 106 (e.g., proximate to a heart 103, proximate to the spinal cord). Additionally or alternatively, the IMD 101 may have components that are external to the patient 106, for example, the IMD 101 may constitute a neuro external pulse generator (EPG). The IMD 101 may be one of various types of implantable devices, such as, for example, an implantable cardiac monitoring device (ICM), a leadless pacemaker, neurostimulator, electrophysiology (EP) mapping and radio frequency (RF) ablation system, an implantable pacemaker, implantable cardioverter-defibrillator (ICD), defibrillator, cardiac rhythm management (CRM) device, an implantable pulse generator (IPG), or the like.

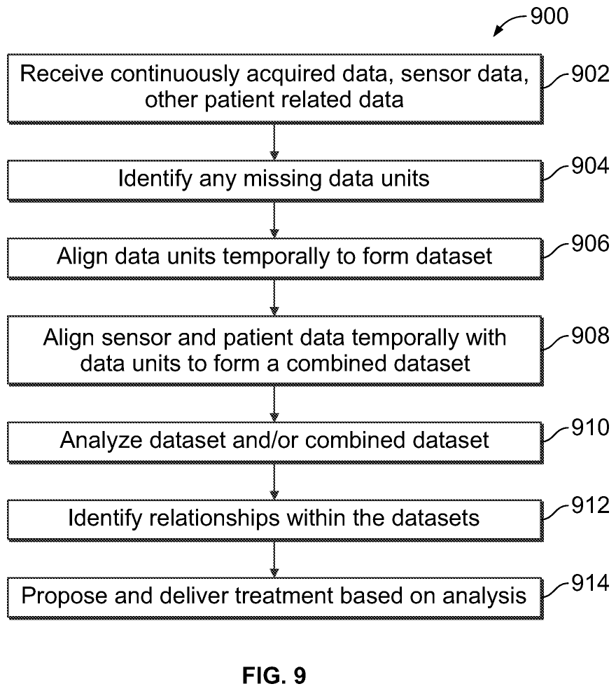

One or more additional IMD and/or physiological sensor 110 can be implanted within and/or be attached to and/or in contact with the skin of the patient 106. The physiological sensor 110, which can be and/or include one or more sensing circuit that includes at least one sensing channel for sensing physiological data, can be any number of medical monitoring sensors, such as an accelerometer, an SpO2 sensor, pulmonary arterial pressure sensor, blood pressure sensor, blood glucose level sensor, sensors for sensing blood components for analysis, and the like. The physiological sensor 110 can sense and acquire data such as acceleration signature, blood glucose levels, pulmonary arterial pressure, movement, posture, activity, oxygen saturation, etc., and convey the acquired data to the external device 108 over communication link 114 (e.g., such as by using a transceiver) and/or the IMD 101 over communication link 116. The communication links 114, 116 can transfer data using the same or similar protocols to the communication link 104 described herein. In some embodiments, the continuously acquired data from the IMD 101 and the data acquired by the physiological sensor 110 can be correlated temporally (as discussed in further detail herein, including but not limited to, in FIG. 9), such as to review EGM data acquired while other certain conditions are occurring, such as when blood glucose level is in a predetermined range.

The patient 106 can keep the external device 108 in relatively close proximity to their person to facilitate communication over communication links 104, 114 and in operating condition, such as with the battery charged, so that the CDCM can be enabled and disabled, and continuously acquired data can be collected and transmitted as desired, as scheduled, etc. In some embodiments, the App 112 is kept open and running on the external device 108. The App 112 can be preprogrammed to enable and disable a particular CDCM at specific times and/or based on specific conditions and/or triggers. In other cases, the patient 106 can interact with the App 112 by selecting from a list of one or more CDCMs, resulting in the automatic or manual enabling and/or disabling of the CDCM. The App 112 can advise the patient 106 that a CDCM is enabled or disabled, that continuously acquired data is being transferred, that the transfer is complete, that the transfer and/or communication link 104, 114 failed and/or was unexpectedly terminated, and the like.

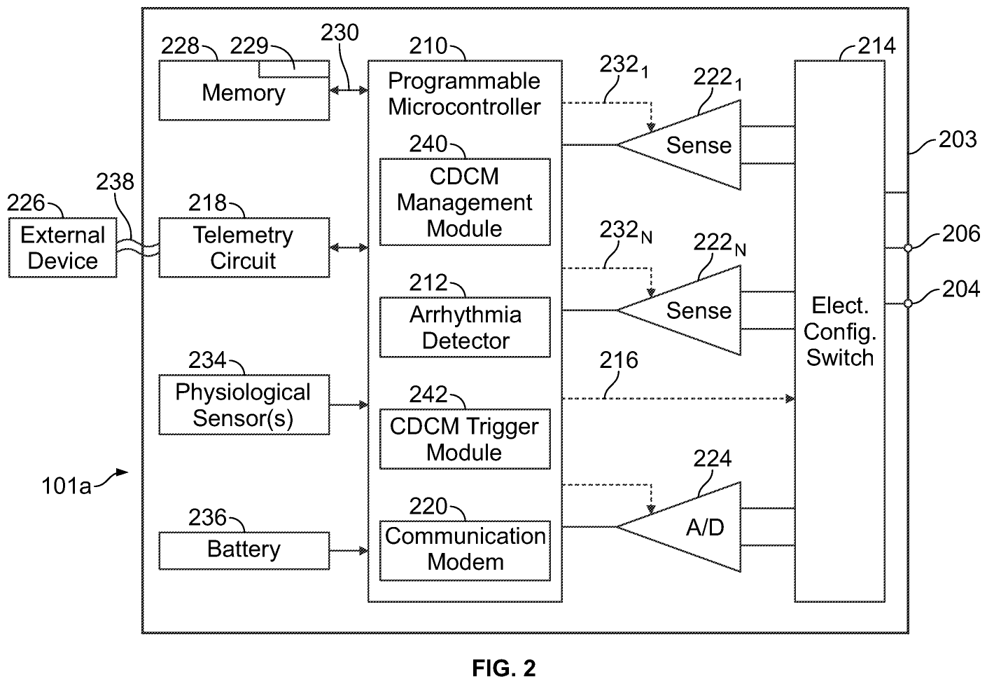

FIG. 2 illustrates a block diagram of an exemplary IMD 101a (generally referred to herein as IMD 101) that is configured to be implanted into the patient 106 in accordance with embodiments herein. The systems described herein can include or represent hardware and associated instructions (e.g., software stored on a tangible and non-transitory computer readable storage medium, such as a computer hard drive, ROM, RAM, or the like) that perform the operations described herein. The hardware may include electronic circuits that include and/or are connected to one or more logic-based devices, such as microprocessors, processors, controllers, or the like. These devices may be off-the-shelf devices that perform the operations described herein from the instructions described above. Additionally or alternatively, one or more of these devices may be hard-wired with logic circuits to perform these operations.

The IMD 101 is configured to collect continuously acquired data, which may include physiologic data and marker data, which can include one or more temporal markers for sensing, timing of QRS, arrhythmia detection and termination, noise, activity, sleep, posture, physiological events, and the like. In some embodiments, other device related data can also be collected and temporally correlated. For example, in connection with collecting physiologic data, the IMD 101 may be implemented to monitor ventricular activity alone, or both ventricular and atrial activity through sensing circuitry. Additionally or alternatively, the IMD 101 may monitor respiratory activity, heart sounds, diabetes related physiologic information, cholesterol, impedance, nerve fiber activity, brainwave activity and the like. The IMD 101 has a housing 203 to hold the electronic/computing components. The housing 203 (which is often referred to as the “can”, “case”, “encasing”, or “case electrode”) may be programmably selected to act as an electrode for certain sensing modes. Housing 203 further includes a connector (not shown) with at least one terminal 206 and preferably a second terminal 204. The terminals 206, 204 may be coupled to sensing electrodes (on the device housing, in the header, or located otherwise) that are provided upon or immediately adjacent the housing 203. Additionally or alternatively, the terminals 206, 204 may be connected to one or more leads having one or more electrodes provided thereon, where the electrodes are located in various locations about the heart. The type and location of each electrode may vary.

In some embodiments, the IMD 101 is configured to be placed subcutaneously utilizing a minimally invasive approach. Subcutaneous electrodes are provided on the housing 203 to simplify the implant procedure and eliminate a need for a transvenous lead system. The sensing electrodes may be located on opposite sides/ends of the device and designed to provide robust episode detection through consistent contact at a sensor-tissue interface. The IMD 101 may be configured to be activated by the patient or automatically activated, such as by a trigger or other notification, in connection with recording continuously acquired data.

The IMD 101 includes a programmable microcontroller 210 that controls various operations of the IMD 101, including physiologic data monitoring and/or device related data logging. Microcontroller 210 includes a microprocessor (or equivalent control circuitry), RAM and/or ROM memory, logic and timing circuitry, state machine circuitry, and I/O circuitry. As one example of physiologic data, the microcontroller 210 performs the operations in connection with collecting cardiac activity data and analyzing the cardiac activity data to identify episodes of interest. As one example of a technique for analyzing cardiac activity, the microcontroller 210 includes an arrhythmia detector 212 that is configured to analyze cardiac activity data to identify potential AF episodes as well as other arrhythmias (e.g., Tachycardias, Bradycardias, Asystole).

The microcontroller 210 performs the operations in connection with collecting device related data. The microcontroller 210 may periodically run a self-diagnostic check for a status of the IMD 101. For example, the microcontroller 210 may run a self-diagnostic check to verify the device is operating correctly every 24 hours. Optionally, the microcontroller 210 may run a self-diagnostic check when a command is communicated by a patient, physician, and the like. The device related data may represent various types of information regarding a therapy delivered by the IMD 101, an operating condition of the IMD (e.g., battery life, temperature, processing power usage, errors, memory available), as well as other device status, operating state, and condition information that may be of interest to log. For example, the microcontroller 210 may run a self-diagnostic check and identify that the battery life of the IMD 101 is nearing expiration.

In accordance with certain embodiments, the electrodes may be directly coupled to sensing circuits in a predetermined hardwired electrode configuration. Alternatively, a switch 214 may be provided, where the switch 214 is managed by the microcontroller 210 to select different electrode configurations. The switch 214 is controlled by a control signal 216 from the microcontroller 210. Optionally, the switch 214 may be omitted and the circuits directly connected to the housing electrode and a second electrode.

The IMD 101 includes sensing circuitry 222 selectively coupled to one or more electrodes that perform sensing operations, through the switch 214 to detect physiologic data indicative of physiologic activity of interest, such as cardiac activity (CA) data. The sensing circuitry 222 may include dedicated sense amplifiers, multiplexed amplifiers, or shared amplifiers that define multiple sensing channels. It may further employ one or more low power, precision amplifiers with programmable gain and/or automatic gain control, bandpass filtering, and threshold detection circuits to selectively sense the physiologic activity of interest. In one embodiment, switch 214 may be used to determine the sensing polarity of the physiologic signal by selectively closing the appropriate switches.

For embodiments that include multiple sensing circuits 222 (e.g., sensing circuit 222₁, . . . 222ₙ), each of the sensing circuits may represent a separate sensing channel in which each sensing channel may receive a control signal 232 from the microcontroller 210. The control signal 232 may cause or include instructions/commands for adjusting one or more parameters, such as threshold voltages. Different sensing channels may also include different dedicated circuitry. For example, different sensing channels may apply different filters for selectively filtering and amplifying R-waves, P-waves, and T-waves.

In other embodiments, the output from a single sensing circuit 222 is provided by the sensing circuit 222 to multiple separate sensing channels. For example, each of the sensing channels may have an independently-controlled sense amplifier or threshold comparator (not shown). In such embodiments, the same output signal may be processed, in parallel, by multiple sensing channels, such as with different thresholds.

The output of the sensing circuitry 222 is connected to the microcontroller 210 which, in turn, determines when to store the physiologic activity data (digitized by the A/D data acquisition system 224) in the memory 228. The sensing circuitry 222 receives a control signal 232 from the microcontroller 210 for purposes of controlling the gain, threshold, polarization charge removal circuitry (not shown), and the timing of any blocking circuitry (not shown) coupled to the inputs of the sensing circuitry.

The IMD 101 is further equipped with a telemetry circuit 218 (e.g., transceiver circuit) and a communication modem (modulator/demodulator) 220 to enable wireless communication. In one implementation, the telemetry circuit 218 and communication modem 220 use high frequency modulation, for example using RF or Blue Tooth telemetry protocols. The telemetry circuit 218 may include one or more transceivers. For example, the telemetry circuit 218 may be coupled to an antenna in the header that transmits communications signals in a high frequency range that will travel through the body tissue in fluids without stimulating the heart or being felt by the patient. The communication modem 220 may be implemented in hardware as part of the microcontroller 210, or as software/firmware instructions programmed into and executed by the microcontroller 210.

By way of example, the external device 226 may represent a portable electronic device (e.g., smart phone, iPad, laptop computer, smart watch, wearable wristband, wearable garment, pillow, blanket, bedside monitor installed in a patient's home and utilized to communicate with the IMD 101 while the patient is at home, in bed or asleep). The external device 226 may be a programmer used in the clinic to interrogate the device, retrieve data and program detection criteria and other features. The external device 226 may be a device that can be coupled over a network (e.g., the Internet) to a remote monitoring service, medical network and the like. The external device 226 facilitates access by physicians to patient data as well as permitting the physician to review real-time ECG signals (e.g., reviewing within minutes or seconds of acquisition) while the signals are being collected by the IMD 101.

The microcontroller 210 is coupled to a memory 228 by a suitable data/address bus 230. The programmable operating parameters used by the microcontroller 210 are stored in memory 228 and used to customize the operation of the IMD 101 to suit the needs of a particular patient. Such operating parameters define, for example, detection rate thresholds, sensitivity, automatic features, arrhythmia detection criteria, activity sensing or other physiological sensors, and electrode polarity, etc. The operating parameters of the IMD 101 may be non-invasively programmed into the memory 228 through a telemetry circuit 218 in telemetric communication via communication link 238 with the external device 226. The telemetry circuit 218 allows intracardiac electrograms and status information relating to the operation of the IMD 101 (as contained in the microcontroller 210 or memory 228) to be sent to the external device 226 through the established communication link 238. The IMD 101 may further include magnet detection circuitry (not shown), coupled to the microcontroller 210, to detect when a magnet is placed over the IMD 101. A magnet may be used by a clinician to perform various test functions of the IMD 101 and/or to signal the microcontroller 210 that the external device 226 is in place to receive or transmit data to the microcontroller 210 through the telemetry circuits 218.

The IMD 101 can further include one or more physiologic sensor 234. Such sensors are commonly referred to (in the pacemaker arts) as “rate-responsive” or “exercise” sensors. The physiological sensor 234 may further be used to detect changes in the physiological condition of the heart, or diurnal changes in activity (e.g., detecting sleep and wake states). Signals generated by the physiological sensors 234 are passed to the microcontroller 210 for analysis and optional storage in the memory 228 in connection with the cardiac activity data, marker data, episode information and the like. While shown as being included within the IMD 101, the physiologic sensor(s) 234 may be external to the IMD 101, yet still be implanted within or carried by the patient. Examples of physiologic sensors might include sensors that, for example, monitor activity, temperature, sense respiration rate, pH of blood, ventricular gradient, position/posture, minute ventilation (MV), and so forth.

In some embodiments, the physiological sensor 234 can be an accelerometer. For example, the accelerometer may be a chip for placement in the IMD 101. In another embodiment, the accelerometer is formed and operates in the manner described in U.S. Pat. No. 6,937,900, entitled “AC/DC Multi-Axis Accelerometer For Determining A Patient Activity And Body Position,” the complete subject matter which is expressly incorporated herein by reference. The IMD 101 may also include one or more processors for implementing algorithms that use accelerometer data. In one example, a diagnosis algorithm can be provided for detecting arrythmias, syncope, fainting, falls, strokes, heart attacks, or the like. In one example, the diagnosis algorithm is the diagnosis algorithm described and disclosed in U.S. published application US20210345935A1, entitled “System For Verifying A Pathologic Episode Using An Accelerometer” filed Mar. 5, 2021, that is incorporated in full by reference herein. In one example, the IMD 101 includes a three-dimensional (3D) accelerometer based posture algorithm, for example, as described and disclosed in U.S. published application 2023/0346258 filed Mar. 30, 2023, entitled “System For Determining Change in Position of an Implanted Medical Device Within an Implant Pocket” that is incorporated in full by reference herein.

The programmable microcontroller 210 further includes a CDCM management module 240. The CDCM management module 240 can store the parameters/conditions associated with one or more CDCM, as defined herein. The CDCM management module 240 may include programmed software/firmware that identifies buffer memory 229 that is temporarily allocated to storing the continuously acquired data during the CDCM operation. The continuously acquired data can include physiologic data such as one or more channels of EGM data, marker data, and/or sensor related data (e.g., detected/determined by physiological sensor(s) 234, data received from a sensor external to the IMD 101, such as from the physiological sensor 110). The buffer memory 229 can be allocated to store only the continuously acquired CDCM data for a predetermined duration of time, predetermined number of data units (discussed further below), until a trigger is received indicating that the CDCM is disabled or terminated, and/or until a predetermined maximum threshold of time is reached.

The programmable microcontroller 210 further includes a CDCM trigger module 242 that can function as an input to receive and/or detect trigger(s), and function as an output to enable and/or disable a CDCM. The CDCM trigger module 242 monitors and/or receives data from various sensors and IMDs, such as the physiological sensor 234 and physiological sensor 110 (FIG. 1), to detect certain features of interest and/or determine whether monitored levels exceed predetermined thresholds. For example, if the physiological sensor 110 monitors blood glucose, the CDCM trigger module 242 can be configured to request and/or receive a current blood glucose level, such as through the communication links 104, 114 and the external device 108. In other embodiments, the IMD 101 and physiological sensor 110 can be configured to communication directly with each other, wherein the physiological sensor 110 can transmit data associated with blood glucose levels directly to the IMD 101. In this example, the physiological sensor 110 can similarly record data that can be transferred to the external device 108 and correlated temporally with the continuously acquired EGM data. In still further embodiments, the CDCM trigger module 242 can monitor and/or receive notifications from the physiological sensor(s) 234 indicating the level of a certain parameter. The CDCM trigger module 242 can determine whether a threshold has been reached, and thus the CDCM associated with the particular physiological feature is to be enabled or disabled.

In accordance with embodiments herein, responsive to receiving a trigger, the programmable microcontroller 210 enables the CDCM that includes a predetermined sampling rate. Once enabled, the physiological characteristic(s), such as but not limited to the CA signals, are continuously sensed and physiological data indicative of the physiological characteristic(s) is generated, such as by sensing circuitry 222, for a duration of a collection session associated with the CDCM, wherein the amount of the CA data and/or EGM data collected during the collection session exceeds a capacity of the buffer memory 229. In some cases, the raw signal sensing may use a higher sample rate, and the CA signals are resampled based on the CDCM instruction and are saved in the buffer memory 229. The IMD 101, such as by using the telemetry circuit 218, connects with the external device 108, and the programmable microcontroller 210 transmits the CA data and/or EGM data saved in the buffer memory 229 to the external device 108. The connect and transmit operations can be performed at a periodic communication interval during the collection session. In some embodiments, the CDCM management module 240 senses the CA signals at the predetermined sampling rate for a predetermined duration. The periodic communication interval can be determined based on one or more of i) a capacity of the buffer memory, ii) a number of the at least one sensing channels configured to sense the CA signals during the collection session, iii) the predetermined sampling rate, iv) an expected connection time needed to establish the connection between the IMD 101 and the external device 108, v) the size or capacity of the memory space of the external device receiving the continuously acquired data, or vi) a data transfer speed between the IMD 101 and the external device 108.

In some embodiments, the CDCM trigger module is configured to detect first and second triggers associated with different CDCMs, wherein in response to the first trigger, the CDCM management module 240 continuously senses the CA signals at a first sampling rate, and in response to the second trigger, the CDCM management module 240 continuously senses the CA signals at a second sampling rate that is different than the first sampling rate. In other embodiments, the CDCM management module 240 continuously senses the CA signals for a first duration, and in response to the second trigger, the CDCM management module 240 continuously senses the CA signals for a second duration that is different than the first duration. The trigger can be a programmed trigger having a predetermined schedule and a predetermined collection session.

In other embodiments, the programmable microcontroller 210 is configured to determine a physiological feature, wherein in response to the physiological feature exceeding a threshold, the CDCM management module 240 disables the CDCM. The physiological feature can be a heart rate and may be sensed by the physiological sensor 234. In still further embodiments, in response to receiving a disabling message (e.g., disabling trigger), such as with the CDCM trigger module, the CDCM management module 240 disables the CDCM.

The CDCM management module 240 connects with the external device 108 at least a first time and a second time, and the telemetry circuit 218 transmits a first set of data during the first time and transmits a second set of data during the second time that is different from the first set of data.

A battery 236 provides operating power to all of the components in the IMD 101. The battery 236 is capable of operating at low current drains for long periods of time. The battery 236 also desirably has a predictable discharge characteristic so that elective replacement time can be detected. As one example, the IMD 101 employs lithium/silver vanadium oxide batteries. The battery 236 may afford various periods of longevity (e.g., three years or more of device monitoring). In alternate embodiments, the battery 236 could be rechargeable. See for example, U.S. Pat. No. 7,294,108, entitled “Cardiac event microrecorder and method for implanting same”, which is hereby incorporated by reference in its entirety.

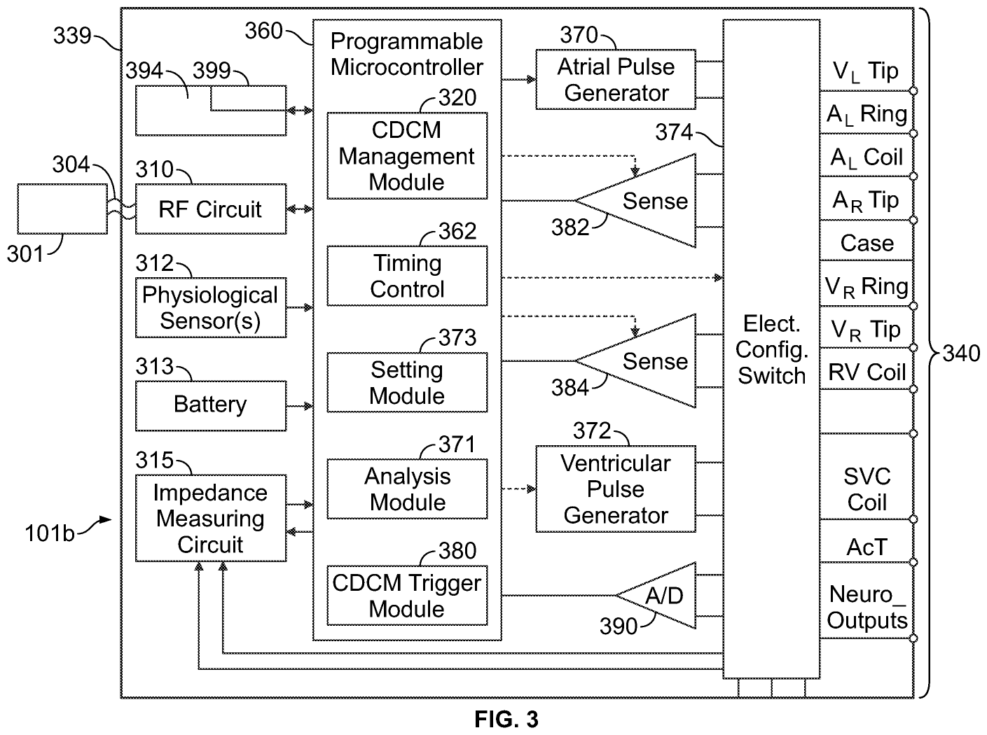

FIG. 3 illustrates another block diagram of internal components of an IMD 101b (generally referred to herein as IMD 101) in accordance with embodiments herein. The IMD 101 is for illustration purposes only, and it is understood that the circuitry could be duplicated, eliminated or disabled in any desired combination to provide a device capable of treating the appropriate chamber(s) with cardioversion, defibrillation and/or pacing stimulation as well as providing for apnea detection and therapy. A housing 339 for the IMD 101, shown schematically in FIG. 3, is often referred to as the “can”, “case” or “case electrode” and may be programmably selected to act as the return electrode for all “unipolar” modes. The housing 339 may further be used as a return electrode alone or in combination with one or more of the coil electrodes for shocking purposes. The housing 339 further includes a connector (not shown) having a plurality of terminals 340 (shown schematically). For convenience, the names of the electrodes are shown next to the terminals. The electrodes can be mounted within and/or proximate the heart. Optionally, the terminals may include an acoustic terminal (ACT) adapted to be connected to an external acoustic sensor or an internal acoustic sensor, depending upon which (if any) acoustic sensors are used. Optionally, the terminals may include a terminal adapted to be connected to a blood sensor to collect measurements associated with glucose levels, natriuretic peptide levels, or catecholamine levels. Optionally, the terminals 340 may include one or more terminals adapted to be connected to nerve fiber sensors.

The IMD 101 includes a programmable microcontroller 360 which controls operations. The microcontroller 360 (also referred to herein as a processor module or unit) typically includes one or more processors or microprocessors, or equivalent control circuitry, designed specifically for controlling the delivery of stimulation therapy and may further include RAM or ROM memory, logic, timing circuitry, state machine circuitry, and I/O circuitry. Typically, the microcontroller 360 includes the ability to process or monitor input signals (data) as controlled by program code stored in memory. The details of the design and operation of the microcontroller 360 are not critical to the embodiments described herein. Rather, any suitable microcontroller 360 may be used that carries out the functions described herein. Among other things, the microcontroller 360 receives, processes, and manages storage of digitized data sets from the various sensors and electrodes. For example, the data sets may include physiologic data such as EGM data, pressure data, heart sound data, and the like. Additionally or alternatively, the data sets may include device related data such as therapy delivery, battery life, available memory, device errors, and the like.

The microcontroller 360 includes the ability to perform the operations of collecting device related data. The microcontroller 360 may periodically run a self-diagnostic check for a status of the IMD 101. For example, the microcontroller 360 may run a self-diagnostic check to verify the device is operating correctly. The device related data may represent various types of information regarding a therapy delivered by the IMD 101, an operating condition of the IMD 101, as well as other device status, operating state, and condition information that may be of interest to log. For example, the microcontroller 360 may run a self-diagnostic check every 24-hours and identify that the battery life of the IMD 101 is nearing expiration. Optionally, the microcontroller 360 may run a self-diagnostic check when a check command is communicated to the IMD 101 by a patient, physician, and the like. The microcontroller may identify the data to be logged as device related data of interest.

The IMD 101 includes one or more pulse generators 370, 372 to generate pacing stimulation pulses for delivery to electrodes via an electrode configuration switch 374. The pulse generators 370, 372 may include dedicated, independent pulse generators, multiplexed pulse generators or shared pulse generators. The pulse generators 370,372 are controlled by the microcontroller 360 via appropriate control signals to trigger or inhibit the stimulation pulses. As one example, the pulse generators 370, 372 may generate atrial and ventricular pacing pulses, cardioversion therapy, defibrillation shocks and the like. Optionally, the IMD 101 may represent a neuro stimulation device, in which case the pulse generators 370, 372 represent neuro pulse generators to generate stimulation pulses for a brain or spinal cord nervous system. In this alternative embodiment, the stimulation pulses are delivered by a plurality of electrodes through a neuro-stimulation lead.

The microcontroller 360 further includes timing control circuitry 362 used to control the timing of stimulation pulses (e.g., pacing rate, atria-ventricular (AV) delay, atrial interconduction (A-A) delay, or ventricular interconduction (V-V) delay, neurostimulation therapy, brainwave therapy). Optionally, the timing control circuitry 362 monitors the timing of the physiologic characteristics of interest, such as refractory periods, blanking intervals, noise detection windows, evoked response windows, alert intervals, marker channel timing, and the like. Switch 374 includes a plurality of switches for connecting the desired electrodes to the appropriate I/O circuits, thereby providing complete electrode programmability. Accordingly, the switch 374, in response to a control signal from the microcontroller 360, determines the polarity of the stimulation pulses (e.g., unipolar, bipolar) by selectively closing the appropriate combination of switches (not shown) as is known in the art.

Sensing circuits 382, 384 are selectively coupled to the leads and/or electrodes through the terminals 340 and switch 374. The sensing circuits are configured to detect various physiologic characteristics of interest and generate physiologic data indicative of the physiologic characteristics. At least a portion of the physiologic data is then stored as data in memory 394. The sensing circuits 382, 384, may include dedicated sense amplifiers, multiplexed amplifiers or shared amplifiers. When implemented in connection with a pacemaker, cardioverter and/or defibrillator, the outputs of the sensing circuits 382, 384 are connected to the microcontroller 360 and are used to trigger or inhibit generation of atrial and ventricular pulses, respectively, in a demand fashion in response to the absence or presence of cardiac activity in the appropriate chambers of the heart.

Physiologic signals are also applied to the inputs of an analog-to-digital (A/D) data acquisition system 390. For example, the data acquisition system 390 is configured to acquire physiologic data signals, convert the raw analog data into a digital physiologic signal, and store the digital physiologic data in memory 394 for later processing and/or RF transmission. In some embodiments the data can be stored in buffer memory 399 prior to being transferred to an external device 301 as explained herein. The data acquisition system 390 is coupled to one or more electrodes and leads through the switch 374 to sample cardiac signals across any combination of desired electrodes. The data acquisition system 390 may also be coupled, through switch 374, to one or more other types of sensors such as acoustic sensors. The data acquisition system 390 may also acquire, perform A/D conversion, produce and save digital pressure data, acoustic data, and the like.

The microcontroller 360 includes an analysis module 371 and a setting module 373 that function in accordance with embodiments described herein. When implemented in a pacemaker, the analysis module 371 analyzes a characteristic of interest from the heart. The level of the characteristic changes as the pacing parameter is changed. The setting module 373 sets a desired value for the pacing parameter based on the characteristic of interest from the heart. The pacing parameter may represent at least one of an AV delay, a VV delay, a VA delay, intra-ventricular delays, electrode configurations and the like. The microcontroller 360 changes at least one of the AV delay, the VV delay, the VA delay, the intra-ventricular delays, electrode configurations and like in order to reduce systolic turbulence and regurgitation. In some embodiments, the IMD 101 of FIG. 3 can also include an arrhythmia detector 212 as shown and discussed in FIG. 2.

An RF circuit 310 (e.g., transceiver circuit) is configured to handle and/or manage the communication link 104, 304 between the IMD 101 and the external device 301. The RF circuit 310 is electrically coupled to the microcontroller 360, and is controlled by the microcontroller 360 and may support a particular wireless communication protocol while communicating with the external device 108, such as BLE, Bluetooth, ZigBee, Medical Implant Communication Service (MICS), or the like. Protocol firmware may be stored in memory 394, which is accessed by the microcontroller 360. The protocol firmware provides the wireless protocol syntax for the microcontroller 360 to assemble data packets, establish communication links 104, 304, and/or partition data received from the external device 301.

The microcontroller 360 is electrically coupled to the memory 394 by a suitable data/address bus, wherein the programmable operating parameters used by the microcontroller 360 are stored and modified, as required, in order to customize the operation of IMD 101 to suit the needs of a particular patient. The memory 394 may be a non-transitory computer readable medium such as RAM, ROM, EEPROM, a hard drive, or the like.

The memory 394 comprises a buffer memory 399, which can be a section or portion of the memory 394 or a separate memory circuit within the IMD 101. The buffer memory 399 records and temporarily stores data sets (raw data, summary data, histograms, etc.), such as one, two, three, or more of the EGM data, heart sound data, pressure data, Sv02 data, blood glucose data, pulmonary artery pressure, pulse oximetry, posture, timing of QRS, arrhythmia detection and termination, noise, activity, sleep, physiological events, posture of patient, and the like for a desired duration (e.g., 30 minutes, 2 hours, 24 hours) as discussed herein. The buffer memory 399 may be configured to continuously cyclically record and store data for the duration of the CDCM, during which time, stored data is periodically transferred to the external device 301. In some embodiments, if the transfer of data is not accomplished prior to filling the buffer memory 399, the data can be overwritten in a first-in-first-out (FIFO) continuous buffer. When the CDCM is not actively acquiring data, the buffer memory 399 may store other data for other analysis and processing. Therefore, in other embodiments, if the CDCM is stopped or disabled, and the last data transfer of the CDCM is completed, any data remaining in the buffer memory 399 is nulled to avoid confusion associated with review of the data.

The microcontroller 360 further includes a CDCM management module 320, similar to the CDCM management module 240, that may include programmed software/firmware that programs the sensing circuitry 382, 384, switch 374, and the like to acquire EGM data over selected channels at predefined frequencies (e.g., 128 Hz, 256 Hz) as discussed herein. The CDCM management module 320 facilitates the acquisition of the EGM data for a predetermined duration, or until a trigger is received to disable the CDCM. In some cases, the CDCM management module 320 periodically initiates communication with the external device 108, 301 to facilitate the transfer of a plurality of data units stored in the buffer memory 399.

The microcontroller 360 further includes a CDCM trigger module 380 similar to the CDCM trigger module 242 discussed herein. The CDCM trigger module 380 monitors and/or receives data from various sensors and IMDs, such as the physiological sensor(s) 312 and physiological sensor 110, to detect certain features of interest and/or determine whether monitored levels exceed predetermined thresholds. For example, if the physiological sensor 110 monitors blood glucose, the CDCM trigger module 380 can be configured to request and/or receive a current blood glucose level, such as through the communication links 104, 114, 304 and the external device 108. In other embodiments, the IMD 101 and physiological sensor 110 can be configured to communication directly with each other, wherein the physiological sensor 110 can transmit data associated with blood glucose levels directly to the IMD 101. In this example, the physiological sensor 110 can similarly record data that can be transferred to the external device 108 and correlated temporally with the continuously acquired EGM data. In still further embodiments, the CDCM trigger module 380 can monitor and/or receive notifications from the physiological sensor(s) 312 indicating the level of a certain parameter. The CDCM trigger module 380 can determine whether a threshold has been reached, and thus the CDCM associated with the particular physiological feature is to be enabled or disabled.

The pacing and other operating parameters of the IMD 101 may be non-invasively programmed into the memory 394 through the RF circuit 310 via the communication link 304. The RF circuit 310 is controlled by the microcontroller 360 and receives data for transmission by an interconnect. The RF circuit 310 allows various types of data (e.g., intra-cardiac electrograms, marker data, pressure data, acoustic data, Sv02 data, and status information relating to the operation of IMD 101 as contained in the microcontroller 360 or memory 394) to be sent to the external device 301 through the established communication link 304. The RF circuit 310 also allows new pacing parameters for the setting module 373 used by the IMD 101 to be programmed through the communication link 304. Examples of establishing the communication link 304 between the external device 301 and the IMD 101 can be found in, for example, U.S. Pat. No. 10,569,092, entitled “Systems and methods for patient activated capture of transient data by an implantable medical device”, and U.S. Pat. No. 9,289,614, entitled “System and method for communicating with an implantable medical device”, which are hereby incorporated by reference in their entireties.

The IMD 101 may also include a physiological sensor 312, such as an accelerometer, commonly referred to as a “rate-responsive” sensor, to record the activity level of the patient or adjust pacing stimulation rate according to the exercise state of the patient. Optionally, the physiological sensor 312 may further be used to detect changes in cardiac output, changes in the physiological condition of the heart, or changes in activity (e.g., detecting sleep and wake states) and movement positions of the patient. While shown as being included within IMD 101, it is to be understood that the physiological sensor 312 may also be external to the IMD 101, yet still be implanted within or carried by the patient. A common type of rate responsive sensor is an activity sensor incorporating an accelerometer or a piezoelectric crystal, which is mounted within the housing 339 of the IMD 101.

The physiological sensor 312 may be used as the acoustic sensor that is configured to detect the heart sounds. For example, the physiological sensor 312 may be an accelerometer that is operated to detect acoustic waves produced by blood turbulence and vibration of the cardiac structures within the heart (e.g., valve movement, contraction and relaxation of chamber walls and the like). When the physiological sensor 312 operates as the acoustic sensor, it may supplement or replace entirely acoustic sensors. Other types of physiologic sensors are also known, for example, sensors that sense the oxygen content of blood, respiration rate and/or minute ventilation, pH of blood, ventricular gradient, etc. However, any sensor may be used which is capable of sensing a physiological parameter that corresponds to the exercise state of the patient and, in particular, is capable of detecting arousal from sleep or other movement.

The IMD 101 additionally includes a battery 313, which provides operating power to all of the circuits shown. The IMD 101 is shown as having impedance measuring circuit 315 which is enabled by the microcontroller 360. Herein, impedance is primarily detected for use in evaluating ventricular end diastolic volume (EDV) but is also used to track respiration cycles. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 315 is advantageously coupled to the switch 374 so that impedance at any desired electrode may be obtained.

Although not shown herein, it should be understood that the sensor 110 (FIG. 1) can also include memory, programmable microcontroller, telemetry circuit, CDCM management module, CDCM trigger module, and the like. The sensor 110 can also include one or more sensing channel(s) for sensing associated physiological data. Therefore, the sensor 110 can similarly enable a CDCM, sense one or more physiological data, store the data, connect with an external device, and transmit the data. The operations of the sensor 110 can be accomplished together with the IMD 101 or without the IMD 101.

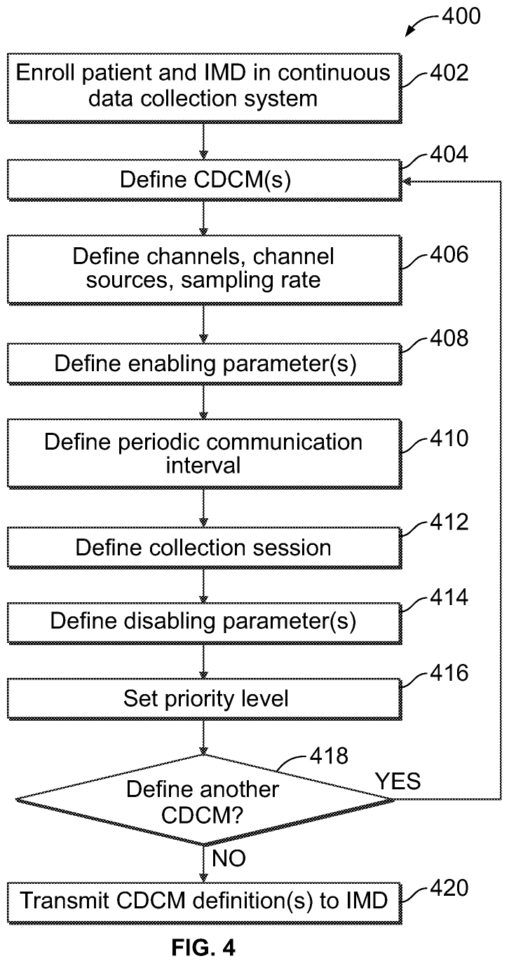

FIG. 4 illustrates a flowchart of a method 400 for programming CDCM(s) for an IMD 101 in accordance with embodiments herein. In some embodiments, a sensor, such as the physiological sensor 110, 234, 312, which can be a pulmonary artery pressure sensor, blood glucose monitoring sensor, or other type of sensor discussed and/or incorporated by reference herein, can be programmed with the CDCM(s), to be used to acquire data simultaneously with the IMD 101 or separately from the IMD 101. The sensor can include one or more processor, memory, telemetry and/or RF circuitry, and the like. At least one technical effect of at least one portion of the methods described herein includes defining parameters associated with one or more CDCM, such as i) number of channels, channel sources, and sampling rate, ii) enabling parameter(s), iii) periodic communication interval, iv) collection session, v) disabling parameter(s), and vi) priority level (optional). The operations of FIG. 4 may be implemented by hardware, firmware, circuitry and/or one or more processors housed partially and/or entirely within the IMD 101, a local external device, remote server or more generally within a health care system. Optionally, the operations of FIG. 4 may be partially implemented by an IMD 101 and partially implemented by a local external device, remote server or more generally within a health care system. For example, the IMD 101 includes IMD memory and one or more IMD processors, while each of the external devices/systems (e.g., local, remote or anywhere within the health care system) include external device memory and one or more external device processors. It should be recognized that while the operations of FIG. 4 are described in a somewhat serial manner, one or more of the operations may be continuous and/or performed in parallel with one another. For example, the various operations of the IMD 101 may be continuous and/or performed in parallel with one another and/or other functions of the IMD 101.

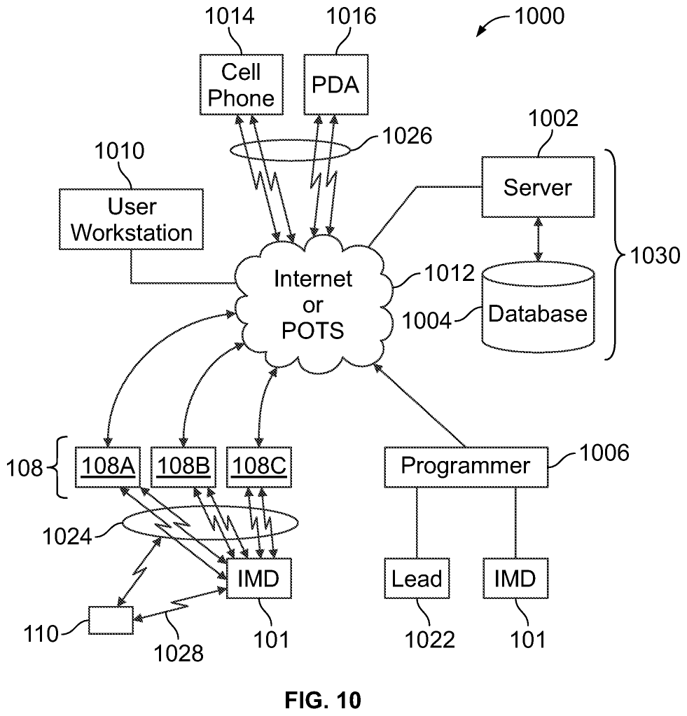

At 402, a practitioner enrolls the patient 106 and the IMD 101 in a continuous data collection system 1030 (FIG. 10). The continuous data collection system 1030 can be housed within one or more database located remote from the practitioner and patient 106 and can be remotely distributed (e.g., the cloud). The practitioner can utilize a user interface (UI) to enter data into an external device 108 such as a phone, tablet, programmer, desktop computer, and the like. In some embodiments, the continuous data collection system 1030 can be housed within and/or interface with a patient care network, such as the Merlin.net™ patient care network operated by Abbott Laboratories (headquartered in the Abbott Park Business Center in Lake Bluff, Ill.). Similar patient care networks, such as that associated with continuous glucose monitoring, can also house and/or interface with the continuous data collection system 1030.

At 404, a first CDCM is defined. In some embodiments, the first CDCM can be identified in the continuous data collection system 1030 as being associated with an activity, such as dialysis or other medical procedure, sleeping, activity, patient identified symptoms, symptom(s) identified by a sensor 110, 234, 312 or the IMD 101, etc. By way of example, a practitioner populates a form or template on the continuous data collection system 1030 for each of the CDCM(s). In some embodiments, forms or templates can be pre-populated by predetermined parameters that the practitioner can accept and/or modify.

At 406, a number of channels is defined (e.g., 1 channel, 2 channels, 3 channels) and EGM channel sources are identified. For example, particular electrode combinations may be identified, certain processing algorithms, etc. For example, single channel=EGM, two channels=EGM+impedance or EGM+heart sound, or three channels=atrial EGM+ventricular EGM+heart sound. It should be understood that other combinations are contemplated, including combinations of channels that do not include acquiring EGM, such as one or more channel that receives physiological data from a sensing circuit that is within the IMD, external to the IMD and within the body, and/or external to the IMD and outside the body. A sampling rate (e.g., 128 Hz, 256 Hz) is defined (e.g., predetermined) for each channel used. In some embodiments, other data is identified to be collected during the CDCM, such as marker data, physiological sensor data, heart rate data, and the like. Such data can be added to the continuously acquired data and stored together in the buffer memory 229, 399.

At 408, parameter(s) to enable (e.g., start) the CDCM are defined. The CDCM can be enabled by a trigger. The trigger can be temporally-based, such as for a specified clock time, particular calendar days and/or days of the week. The trigger can be set and/or sent when the IMD 101 and/or other sensor senses arrhythmia, elevated heart rate, increased activity, blood glucose levels, etc. The trigger can be received from a physiological sensor 110, 234, 312, from the external device 108, a programmer, or other remote device, etc. In some embodiments, the patient 106 can enable the CDCM, such as by selecting from one or more CDCMs displayed within the App 112, when the patient 106 experiences symptoms, and/or when engaging in a particular activity (e.g., dialysis, treatment, medication, exercise). In some embodiments, the CDCM is enabled immediately upon receiving an instruction, while in other embodiments, the CDCM is enabled after a predetermined time delay. In some embodiments, more than one parameter is defined to enable the CDCM, such as a time of day and an instruction from the external device 108. In some cases, the CDCM trigger module 242, 380 can control the enabling and disabling of the CDCM.

At 410, a periodic communication interval is defined for retrieving/transmitting the continuously acquired data. The time between retrievals/transmissions can be based on expected connection time between the IMD 101 and the external device 108, the size of the buffer memory 229, 399 allotted to store the continuously acquired data, the number of channels used, sampling rate, the size or capacity of the memory space of the external device 108 receiving the continuously acquired data, and/or a data transfer speed between the IMD 101 and the external device 108. In other embodiments, the periodic communication interval can be defined when the CDCM is enabled. For example, memory capacity on the external device 108 may be different than that of an external programmer, and in some cases the periodic communication interval can be extended to a maximum to conserve battery power of the IMD 101.

At 412, a collection session (e.g., duration and/or frequency of continuously acquiring data) is defined. The collection session can be continuous, and may be a single collection session, or periodical, wherein multiple collection sessions are repeated over time. A collection session can be defined, such as over a specified number of minutes, hours, or days (e.g., two hours, six hours, 24 hours, two days), intervals per day, intervals per week, intervals per trigger, etc. In some embodiments, the CDCM can have a predetermined schedule, wherein a schedule can be created for enabling and disabling continuous data collection periodically (e.g., between certain clock hours every day for one week, one month, or one year; a predefined number of intervals per day, per week, per month, or per trigger). For example, if a patient undergoes dialysis each Tuesday starting at 10:00 am, such as to evaluate creatinine levels, a collection session may be set to start at or slightly before 10:00 am and extend to include the time dialysis is expected to occur, such as for five hours. In another embodiment, the patient 106 can be monitored for heart failure (HF) over several months or years. In this example, the collection session may be set to extend over two to three hours during sleep in order to filter out the activity related EGM changes. In still further embodiments, continuous collection sessions can be associated with particular triggers, such as arrhythmia, elevated heart rate, increased activity, external command, etc.

At 414, parameter(s) to disable (e.g., end) the CDCM are defined. For example, a programmed trigger can be defined, such as a predetermined end time, predetermined duration, a trigger from a sensor 110, 234, 312, a trigger received from the external device 108, such as a patient selection via the App 112, a trigger received from the external device 108 that was not initiated by the patient or other input on the external device 108, such as a trigger conveyed over the internet to the external device. In some embodiments, a trigger is set to disable the CDCM after a predetermined number of transmissions, and/or after a predetermined number of triggers (e.g., capping the number of times the CDCM is enabled per day, per week, etc., for the same symptoms), etc. In some embodiments, more than one parameter to disable the CDCM is defined. In other embodiments, the CDCM may include the battery level or battery usage of the IMD 101 as a disabling parameter.

Optionally, at 416 a priority level is set for the CDCM. The priority level can be used to resolve conflict between multiple CDCM definitions. For example, a first CDCM may have a low priority and a second CDCM may have a medium or high priority. If the first CDCM is currently enabled and continuously acquiring data when the second CDCM is enabled, the CDCM management module 240, 320 disables the first CDCM and configures the IMD 101 to continuously acquire data based on the definition of the second CDCM. In some embodiments, the data in the buffer memory 229, 399 is transmitted to the external device 108 before being overwritten, while in other embodiments the data in the buffer memory 229, 399 is nulled. In other cases, if the second CDCM is currently enabled and the trigger is received to enable the lower priority first CDCM, the request to enable the lower priority first CDCM is rejected and notified back to user.

At 418, if another CDCM is to be defined, flow returns to 404.

At 420, in some embodiments, one or more processors transmit the one or more CDCM definitions to the IMD 101. For example, the practitioner using the external device 108 can establish communication with the IMD 101 and transmit data collected above (e.g., 404-416). The information associated with the CDCM(s) can be stored, for example, in the memory 228, 394, the CDCM management module 240, 320, CDCM trigger module 242, 380, and/or similar memories and/or modules of the applicable physiological sensor 110, 234, 312.

In some embodiments, the mode definitions (e.g., 420) may be accomplished each time the CDCM is enabled. In this example, the parameters or definition of the mode may not be stored long-term by the IMD 101 or other physiological sensor. Instead, the IMD 101 can receive the mode defining information and be directed to enable continuous acquisition of EGM data and/or other data from the external device 108.

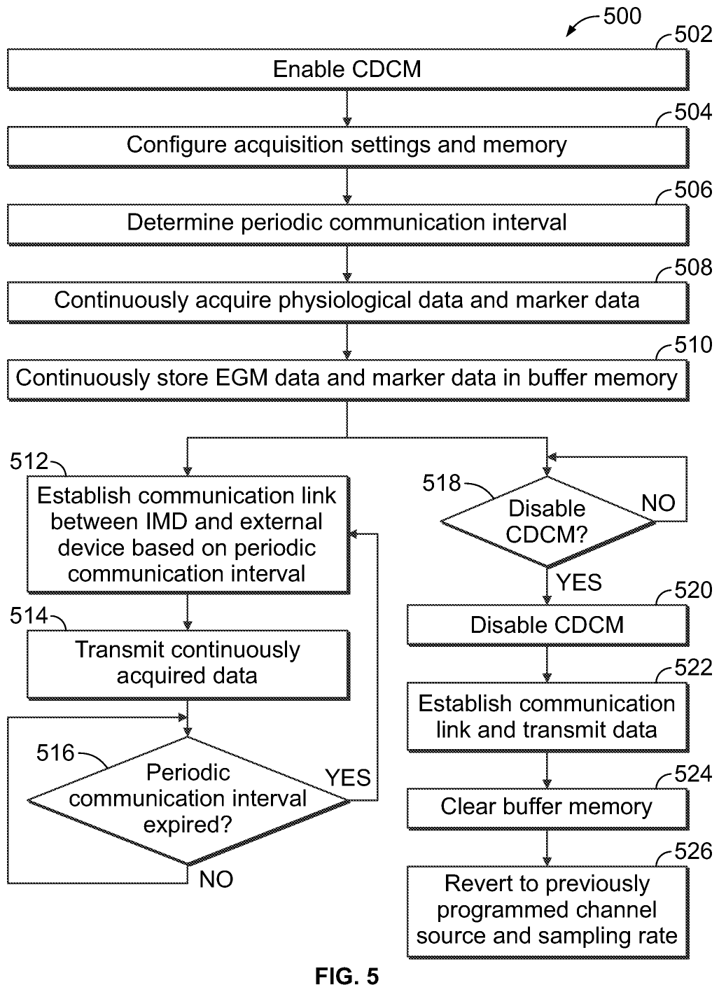

FIG. 5 illustrates a flowchart of a method 500 from the perspective of the IMD 101 for enabling the CDCM and acquiring continuous EGM data in accordance with embodiments herein. At least one technical effect of at least one portion of the methods described herein includes enabling the CDCM, continuously acquiring EGM and/or other data as defined by the CDCM, periodically transferring the continuously acquired data to the external device 108, and disabling the CDCM. The operations of FIG. 5 may be implemented by hardware, firmware, circuitry and/or one or more processors housed partially and/or entirely within the IMD 101, a local external device, remote server or more generally within a health care system. Optionally, the operations of FIG. 5 may be partially implemented by an IMD 101 and partially implemented by a local external device, remote server or more generally within a health care system. For example, the IMD 101 includes IMD memory and one or more IMD processors, while each of the external devices/systems (e.g., local, remote or anywhere within the health care system) include external device memory and one or more external device processors. It should be recognized that while the operations of FIG. 5 are described in a somewhat serial manner, one or more of the operations may be continuous and/or performed in parallel with one another. For example, the various operations of the IMD 101 may be continuous and/or performed in parallel with one another and/or other functions of the IMD 101. Although the operations of FIG. 5 are discussed from the perspective of the IMD 101, it should be understood that similar operations may be accomplished by a sensing circuit, such as the physiological sensor 110.

At 502, one or more processors enable the CDCM on the IMD 101. In some embodiments, the IMD 101 receives a communication from the external device 108 identifying the specific CDCM and an instruction to enable or turn the mode on (e.g., a trigger). As discussed herein, the definition of the CDCM may be stored in the IMD 101 and/or may be transmitted from the external device 108 to the IMD 101. In other embodiments, one or more processors of the IMD 101 detect a trigger, such as a physiological trigger from a physiological sensor 234, 312, a trigger from the physiological sensor 110, and/or detect a physiological condition that initiates the CDCM on the IMD 101. Alternatively or optionally, in cases in which the CDCM is not enabled by the external device 108, the IMD 101 can initiate a communication session with the external device 108 to advise the external device 108 that the CDCM is enabled.

In some embodiments, the patient 106 or practitioner can select the CDCM on the external device 108. One or more CDCM may be available to the patient 106, and may be associated with symptoms for an underlying medical condition such as tachycardia, bradycardia, etc., and activities/functions such as taking of medication, resting, activities such as running, and the like. For example, the patient 106 activates the CDCM through a touch icon on the device screen, speaking to a voice recognition application, and/or shaking and/or moving the external device 108 in certain ways to activate the App 112. Alternatively or additionally, the patient may use a touch function key on a computer, or a touch button icon on a smart watch and/or user wearable electronic accessory such as a Fitbit device to activate the application. The CDCM may be activated by other methods as well. For example, the CDCM can be enabled remotely by a practitioner or automatically based on a schedule. If generated remotely, the enabling communication is sent to the external device 108 near the patient 106, and is then conveyed to the IMD 101.

At 504, one or more processors configure the acquisition settings and the buffer memory 229, 399 of the IMD 101 to acquire continuous data. For example, the specific channels to be used to acquire the continuous EGM data are set along with the sampling rate. The buffer memory 229, 399 is allotted. In some embodiments, any data currently stored in the buffer memory 229 is cleared, invalidated, etc.

At 506, one or more processors determine a periodic communication interval of time at which the IMD 101 and external device 108 should establish communication and transfer the continuously acquired data. In some cases, the periodic communication interval can be determined when the CDCM is defined as discussed herein. In some embodiments, the periodic communication interval is based on one or more of i) the size of the buffer memory 229, 399, ii) number of channels, iii) sampling rate, iv) time lapse expected to occur while establishing the connection between the IMD 101 and the external device 108, v) capacity of the memory space of the external device 108 that will receive and store (e.g., temporarily store) the continuously acquired data, vi) an amount of other data to be acquired and stored in the buffer memory 229, 399 (e.g., sensor data, marker data, physiological data), and/or vii) a data transfer speed between the IMD 101 and the external device 108. For example, in some cases the amount of buffer memory 229, 399 may not always be the same, and the capacity of the memory space of the external device 108 may change (e.g., vary within the same device 101 over time, be different between different ones of the external devices 108). Over the same length of time, CDCM configurations that use more than one channel require more storage than CDCM configurations that use one channel. Similarly, over the same length of time, CDCM configurations that use a higher sampling rate (e.g., 256 Hz) require more storage than CDCM configurations that use a lower sampling rate (e.g., 128 Hz). In other embodiments, the periodic communication interval is predetermined when the CDCM is defined. As discussed elsewhere herein, the periodic communication interval is determined so that the continuous data can be transferred from the IMD to the external device 108 with no loss of the continuous data over time. In some cases, the periodic communication interval is determined to maximize the amount of data sent during each transfer and thus minimize the battery power used by the IMD 101 to establish the connection.

At 508, one or more processors continuously acquire physiological data, such as by sensing physiological characteristic(s) and generating physiological data indicative of the physiological characteristic(s). In some embodiments, the continuously acquired physiological characteristic can be CA data, CA signals, EGM data, blood glucose data, pulmonary artery pressure, pulse oximetry, temperature, heart rate, impedance, blood pressure, blood oxygen saturation, activity, posture, and/or marker data. Marker data can be, but is not limited to, markers indicating the timing of sensing, timing of QRS, arrhythmia detection and termination, noise, activity, sleep, physiological events, posture of patient, etc. The one or more processors continuously acquire the physiological data for the duration of the collection session.

In some embodiments, the IMD 101 continues to acquire other data as programmed and continues to monitor other features, such as arrhythmia (episode detection, discriminators), activity and posture, sleep detection, heart rate variability, noise, magnet detection, battery voltage measurements, etc. In other embodiments, certain functionality will be terminated or suspended when the CDCM is enabled.

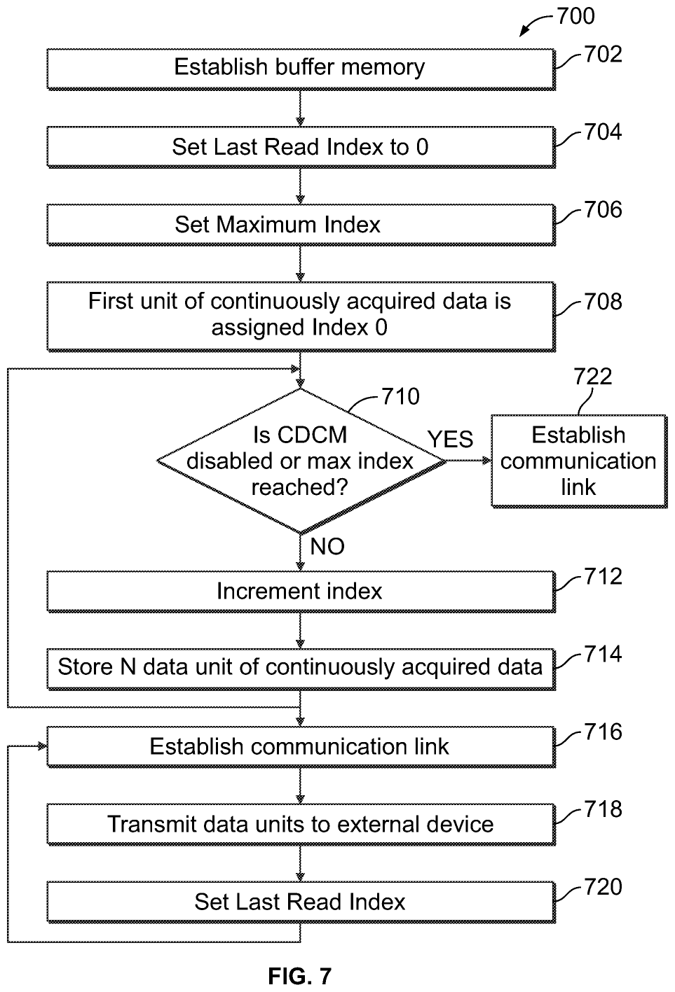

At 510, one or more processors continuously store the continuously acquired data in the buffer memory 229, 399, at the predetermined sampling rate as described further below in FIG. 7. Flow passes simultaneously from 510 to 512 and 518.

At 512, one or more processors periodically establish communication link 104 to connect with the external device 108 (e.g., at periodic communication interval). For example, communication can be initiated by either the IMD 101 or the external device 108. In some embodiments, the external device 108 communicates a request to the IMD 101 to transmit the continuously acquired data.

At 514, one or more processors transmit the continuously acquired data stored in the buffer memory 229, 399 (e.g., set of data) from the IMD 101 to the external device 108. The transfer of data can be accomplished as a “push” from the IMD 101 to the external device 108 or a “pull” initiated by the external device 108. As discussed further below, the EGM and marker data can be acquired, stored, and transferred in a data unit, as discussed elsewhere herein. A partially acquired data unit can be retained in the buffer memory 229 to be transmitted at the next periodic communication interval. In some embodiments, all of the continuously acquired data can be stored in the buffer memory and transmitted in a single transfer operation. When the transfer is complete, the communication between the IMD 101 and the external device 108 can be terminated.

At 516, one or more processors determine whether the periodic communication interval has expired. If yes, the flow returns to 512 to establish communication and transfer the continuously acquired data stored in the buffer memory. For example, the next set of data, which was sensed and stored subsequent to the previous set of data, is transmitted.

At 518, one or more processors determine whether the CDCM should be disabled. For example, one or more processors can determine if a predetermined duration or maximum duration has expired and/or the external device 108 has sent a command to disable the mode (e.g., disabling message, disabling trigger). In other embodiments, one or more processors can determine whether certain physiological parameters are present, have exceeded a threshold, etc., at which time the CDCM should be disabled. For example, if the CDCM is associated with high heart rates and the patient's heart rate falls below/exceeds a predetermined threshold, one or more processors can determine that the CDCM should be disabled. In another embodiment, the CDCM may be associated with blood glucose levels, oxygen saturation levels, etc., and can be disabled when the measured level falls below/exceeds a predetermined threshold. It should be understood that other parameters, thresholds, etc., can be evaluated to dynamically extend or shorten the acquisition duration of the CDCM.

If the CDCM should be disabled, flow passes to 520. At 520, one or more processors disable the CDCM in the IMD 101. At 522, in some embodiments, one or more processors establish the communication link 104, 238, 304, between the IMD 101 and the external device 108 (512) and transmit the continuously acquired data (514).

At 524, one or more processors clear or invalidate any remaining continuously collected data from the buffer memory 229, 399. The buffer memory 229, 399 is now available to store other data acquired by other processes/protocols.

At 526, one or more processors revert the IMD 101 back to the previously programmed channel source and sampling rate, and in some cases may reinitiate some suspended processes.

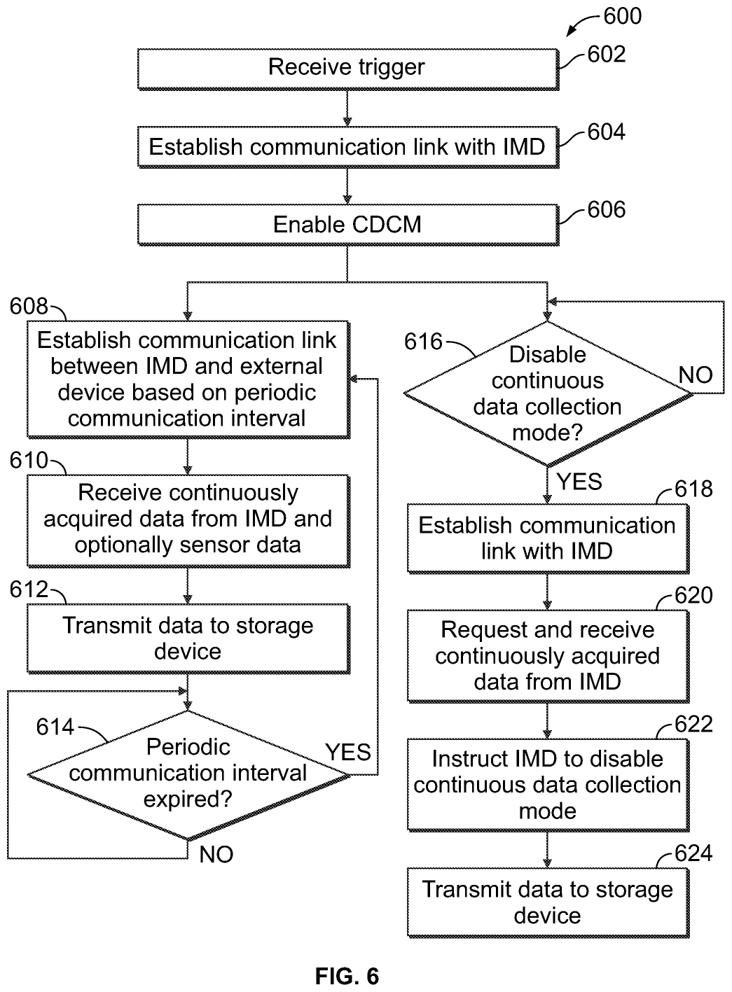

FIG. 6 illustrates a flowchart of a method 600 from the perspective of the external device 108 for enabling the CDCM on the IMD 101 and initiating the acquisition of continuously acquired data in accordance with embodiments herein. At least one technical effect of at least one portion of the methods described herein includes enabling the CDCM, establishing the communication link 104 with the IMD 101, receiving the continuously acquired data from the IMD 101, and disabling the CDCM. The operations of FIG. 6 may be implemented by hardware, firmware, circuitry and/or one or more processors housed partially and/or entirely within the IMD 101, a local external device, remote server or more generally within a health care system. Optionally, the operations of FIG. 6 may be partially implemented by an IMD 101 and partially implemented by a local external device, remote server or more generally within a health care system. For example, the IMD 101 includes IMD memory and one or more IMD processors, while each of the external devices/systems (e.g., local, remote, or anywhere within the health care system) include external device memory and one or more external device processors. It should be recognized that while the operations of FIG. 6 are described in a somewhat serial manner, one or more of the operations may be continuous and/or performed in parallel with one another. For example, the various operations of the IMD 101 may be continuous and/or performed in parallel with one another and/or other functions of the IMD 101.

At 602, one or more processors receive a trigger. The trigger can be preprogrammed from the App 112, such as based on a preset time associated with a CDCM. In other cases, the trigger can be from a user, such as the patient 106, who selects a CDCM from one or more available CDCMs. The patient 106 can further select an option to enable the mode. In other embodiments, the App 112 can receive a communication from a sensor, such as the sensor 110, that triggers the App 112 to initiate enabling a CDCM.

At 604, one or more processors initiate and establish the communication link 104 with the IMD 101. At 606, one or more processors enable the CDCM, such as by sending a command to the IMD 101. It should be understood the external device 108 can also transmit acquisition settings as discussed elsewhere herein. In some embodiments, the IMD 101 can determine and transmit the periodic communication interval to the external device 108. In some embodiments, the App 112 on the external device 108 can display to the patient that the communication link 104 has been established, and that the CDCM is enabled.

Once the required information is exchanged, the communication link 104 between the external device 108 and the IMD 101 is or can be terminated. Once the CDCM on the IMD 101 is enabled, flow passes to both 608 and 616 simultaneously.

Turning first to 608, one or more processors periodically establish the communication link 104 to connect with the IMD 101 (e.g., at periodic communication interval). For example, communication can be initiated by either the IMD 101 or the external device 108. In some embodiments, the external device 108 communicates a request to the IMD 101 to transmit the continuously acquired data from the buffer memory 229, 399 to the external device 108. In some embodiments, the App 112 on the external device 108 can display to the patient that the communication link 104 has been established.

At 610, one or more processors receive the continuously acquired data stored in the buffer memory 229, 399 from the IMD 101. The transfer of data can be accomplished as a “push” from the IMD 101 to the external device 108 or a “pull” initiated by the external device 108. When the transfer is complete, the communication link 104 between the IMD 101 and the external device 108 is terminated or disabled. In some cases, the App 112 can display to the patient 106 that the continuously acquired data is being transferred from the IMD 101 to the external device 108, and also advise when the communication link 104 has been terminated.

In some embodiments, one or more processors of the external device 108 also receive data from sensors, such as the sensor 110, 234, 312. The sensor data can be correlated in time with the continuously acquired data.

At 612, optionally, one or more processors transmit the continuously acquired data that was received to a storage device, such as in the continuous data collection system 1030. For example, the storage device can be a database associated with a physician's office, remote monitoring and analysis program, a manufacturer, etc. In some embodiments, the continuously acquired data is transmitted periodically, such as after every 2, 3, or more receipts of continuously acquired data, or only once after the CDCM is disabled and all data has been received by the external device 108.

At 614, one or more processors determine whether the periodic communication interval has expired. If yes, the flow returns to 608 to establish the communication link 104 and transfer the continuously acquired data.

Turning to 616, one or more processors determine whether the CDCM should be disabled or is disabled. In some embodiments, the CDCM continuously collects data for a predetermined duration. In some cases, when the predetermined duration expires the IMD 101 automatically disables the mode, while in other cases the external device 108 transmits a disable message to the IMD 101. In other embodiments, the one or more processors can determine if the patient 106 has selected to disable the mode, such as through the App 112 or if the mode has been disabled through a different external signal, such as from a remote terminal. In some cases, the patient 106 may enable a CDCM in association with the start of another procedure (e.g., dialysis), and then end the continuous data collection when the procedure is complete or at another desired time. In other embodiments, one or more processors can determine whether the external device 108 has been notified that certain physiological parameters are present, have exceeded a threshold, etc., at which time the CDCM should be disabled. For example, if the CDCM is associated with blood glucose level, and the external device 108 has been informed by the patient 106 or a device, such as an implantable device or sensor 110, that the blood glucose level is below a predetermined threshold, the one or more processors can determine that the CDCM should be disabled. In yet further embodiments, if one or more processors determine that a maximum duration of time has been reached, the CDCM should be disabled.

At 618, one or more processors establish the communication link 104 with the IMD 101 as discussed herein. At 620, one or more processors optionally request and receive continuously acquired data from the IMD 101 and, optionally, sensor data. The App 112 on the external device 108 can display to the patient that the communication link 104 has been established, and that the continuously acquired data is being transferred from the IMD 101 to the external device 108.

At 622, one or more processors instruct the IMD 101 to disable the CDCM. The App 112 on the external device 108, such as on the display, can indicate that the CDCM is disabled. The communication link 104 between the external device 108 and the IMD 101 can be terminated. In some embodiments, the App 112 on the external device 108 can display to the patient that the CDCM is disabled.

At 624, one or more processors transmit the continuously acquired data and sensor data that was received to the storage device as discussed in 612.

FIG. 7 illustrates a flowchart of a method 700 for managing the storing of the continuously acquired data by the IMD 101 and transferring of the continuously acquired data between the IMD 101 and the external device 108 in accordance with embodiments herein. At least one technical effect of at least one portion of the methods described herein includes ensuring that duplicate continuously acquired data is not read, which would unnecessarily consume battery power, assist with data stitching of multiple data units into continuous section(s) of data, and provide a method for identifying gaps in the data and time durations of the gaps. For example, data indexing can be used. The operations of FIG. 7 may be implemented by hardware, firmware, circuitry and/or one or more processors housed partially and/or entirely within the IMD 101, a local external device, remote server or more generally within a health care system. Optionally, the operations of FIG. 7 may be partially implemented by an IMD 101 and partially implemented by a local external device, remote server or more generally within a health care system. For example, the IMD 101 includes IMD memory and one or more IMD processors, while each of the external devices/systems (e.g., local, remote, or anywhere within the health care system) include external device memory and one or more external device processors. It should be recognized that while the operations of FIG. 7 are described in a somewhat serial manner, one or more of the operations may be continuous and/or performed in parallel with one another. For example, the various operations of the IMD 101 may be continuous and/or performed in parallel with one another and/or other functions of the IMD 101.

At 702, one or more processors establish the location(s) of the buffer memory 229, 399 and suspend the ability for other processes to store data in the buffer memory 229, 399. The buffer memory 229, 399 can be a circular, FIFO memory, in which data units of continuously acquired data are written. When the buffer memory 229, 399 is full, the one or more processors will overwrite the oldest acquired data.

In some embodiments, data indexing can be used to facilitate tracking of the stored and retrieved data. It should be understood that other methods of data indexing are contemplated. At 704, one or more processors set a last read index to 0. The last read index represents the last data unit (e.g., a block of continuously acquired EGM data and, in some cases, marker data) that was read by the external device 108. As discussed further below, the external device 108 will update the last read index depending upon the transfer of the data units.

At 706, one or more processors set a maximum index to a maximum number of data units to be acquired during a current CDCM session. The maximum number of data units may vary depending upon the CDCM. The maximum index is compared to the number of data units that are acquired during the current CDCM session to prevent the continued use of device resources (e.g., battery depletion) of the IMD 101 in the event that the CDCM is not disabled by the IMD 101 or the external device 108. In some embodiments, the maximum index can be set to the number of data units equivalent to a maximum duration or amount of time (e.g., 24 hours, 36 hours, 72 hours).

At 708, one or more processors store continuously acquired data in the buffer memory 229, 399 and assign a first data unit of the continuously acquired data an Index of 0.

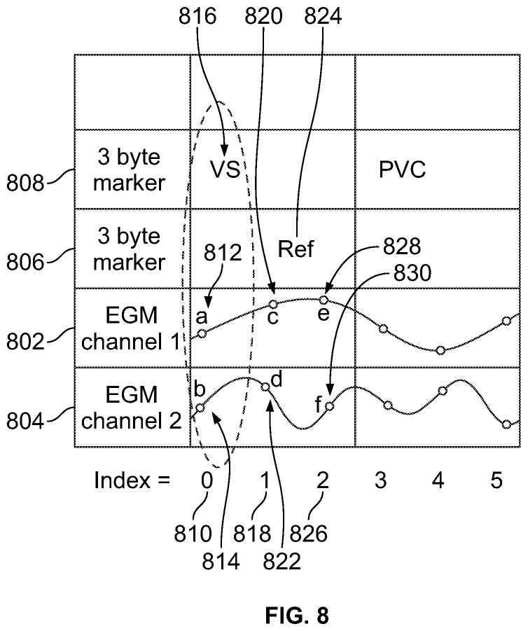

Turning to FIG. 8, this figure is a graphical depiction of several data units of continuously acquired data that are assigned consecutive Index numbers in accordance with embodiments herein. If one EGM channel is enabled, a data unit includes an EGM sample at a specific time point plus the marker data that follows the sample. In the illustrated example, two EGM channels are enabled. The data unit includes one EGM sample from each channel at the same temporal location plus the marker data that follow the EGM sample. In some cases, the data unit can include EGM sample(s) but no marker data, if there are no marker data between consecutive EGM samples. For example, EGM channel 1 802 and EGM channel 2 804, and marker data 806, 808 are continuously acquired and stored in the buffer memory 229, 399. In some cases, the CA data is continuously sensed at a higher sample rate, and the EGM signals are resampled based on the CDCM instruction. FIG. 8 shows six data units; however, the number of data units that can be stored in the buffer memory 229, 399 depends upon the size of the buffer memory 229, 399, the number of channels, etc.

Index 0 810 includes the EGM channel 1 802 sample “a” 812, the EGM channel 2 804 sample “b” 814, and ventricular sense (VS) marker 816 that follows the samples. Index 1 818 includes sample “c” 820, sample “d” 822, and Ref marker 824 (refractory marker that stores information such as EGM peak amplitude during the refractory period, whether the sensed event is interrupted by noise, etc.) that follows the samples. Index 2 826 includes sample “e” 828 and sample “f” 830, and does not include a marker. In some embodiments, one or more of the data units include CA data that may include data other than EGM data.

Returning to FIG. 7, at 710 one or more processors determine whether the CDCM is disabled or the maximum index has been reached. For example, the last stored Index can be compared to the maximum index. If more continuous data is to be collected, at 712 one or more processors increment the Index and at 714 store N data unit of continuously acquired data in the buffer memory 229, 399.

Flow simultaneously returns to 710 to verify whether more continuously acquired data should be/will be acquired or whether the CDCM should be disabled, and to 716, where one or more processors establish a communication link 104 to connect with the external device 108. The communication link 104 can be initiated by the external device 108 and/or the IMD 101. The communication link 104 can be initiated based on a length of time, such as the periodic communication interval.

At 718, one or more processors transmit at least a portion of the data units that are stored in the buffer memory 229, 399 (e.g., a set of data) to the external device 108. The transfer operation can be a push or pull operation. In some embodiments, multiple data units are transmitted from the IMD 101 to the external device 108. Only complete data units are transmitted, such that the indexing method can accurately track and record the transfer and storage of data.

The one or more processors determine what data units to transfer based on the last read index. If the last read index is zero (e.g., this is the first data transfer of the current CDCM), the transfer includes the first index (e.g., Index 0) and a number of additional data units. In some cases, the number of data units can be the maximum number of complete data units. For example, if the IMD 101 has acquired and stored data units 0-25, 26 data units are transferred. In other embodiments, a predetermined number of data units can be transferred, wherein the predetermined number of data units can be less than the maximum number of data units available for transfer. The predetermined number of data units can be different based on the amount of data acquired within each data unit (e.g., one EGM channel, two EGM channels).

At 720, one or more processors set the last read index. For example, if the first index of the continuously acquired data is “i” and the number of data units successfully received by the external device 108 is “k”, the external device 108 sets the last read index to (i+k−1), and transmits the updated last read index to the IMD 101. Accordingly, this indexing allows for gaps in collected data to be identified during post processing. A gap in data is identified if the first Index of the data units retrieved, “i”, in the (n+1)th transmission is greater than (i+k) from the nth transmission. As discussed herein, a gap in data can occur if the external device 108 was not in proximity to the patient 106 for a period of time or otherwise unable to connect with the IMD 101, such as being powered off, lack of battery power, and the like.

If there is a telemetry break in the middle of the retrieval of the continuously acquired data, the last read index may not be updated by the external device 108. In some cases, the external device 108 will initiate another communication session and once connected, will continue to transfer data units from the point of interruption.

The process can return to 716 to establish the next communication session when the periodic communication interval has expired. Accordingly, the one or more processors simultaneously store the continuously acquired data in the data units in an indexed manner while transferring sets of data of the continuously acquired data at a rate that prevents the continuously acquired data from being overwritten in the buffer memory 229, 399 prior to transfer.

Returning to 710, if the CDCM is disabled or the maximum index is reached, flow passes to 722 to establish another communication link 104. In some embodiments, the IMD 101 may initiate the communication link 104. This can facilitate the transfer of the final data units to the external device 108 before the buffer memory 229, 399 is nulled or used to store other data.

FIG. 9 illustrates a flowchart of a method 900 for disease diagnostics and determining EGM based features for biomarker estimate utilizing the continuously acquired data in accordance with embodiments herein. At least one technical effect of at least one portion of the methods described herein includes stitching the multiple data units together to form a combined dataset that is continuous, and identifying any breaks in the continuously acquired data. The combined dataset is used to diagnose disease, determine worsening of an existing disease, detect existing and impending arrhythmia, etc. Further, additional data, such as sensor data and/or temporally aligned data entered by the patient and/or practitioner can be used to identify EGM-based features for biomarker estimate. The operations of FIG. 9 may be implemented by hardware, firmware, circuitry and/or one or more processors housed partially and/or entirely within the IMD 101, a local external device, remote server or more generally within a health care system. Optionally, the operations of FIG. 9 may be partially implemented by an IMD 101 and partially implemented by a local external device, remote server or more generally within a health care system. For example, the IMD 101 includes IMD memory and one or more IMD processors, while each of the external devices/systems (e.g., local, remote or anywhere within the health care system) include external device memory and one or more external device processors. It should be recognized that while the operations of FIG. 9 are described in a somewhat serial manner, one or more of the operations may be continuous and/or performed in parallel with one another. For example, the various operations of the IMD 101 may be continuous and/or performed in parallel with one another and/or other functions of the IMD 101.

First, the physiologic data can be processed to improve disease diagnosis. For example, the physiologic data can facilitate diagnosing an existing disease(s) that is worsening, and/or detection of existing and impending arrhythmia. Types of disease that may be diagnosed include, but are not limited to, heart failure, sleep apnea, renal failure, diabetes, hyper/hypo-tension, cerebrovascular accident, cardiac ischemia including acute coronary syndrome, valvular disease, cardiac myopathies, etc. Arrhythmia detections include PVCs, VT/VF, AF, Aflutter, PACs, sinus tachycardia, etc. Relevant features such as RR intervals, RR interval variabilities, QRS morphologies, QT durations, T wave morphologies, P wave morphologies, PR intervals, frequency contents of the EGMs, or the raw EGM morphologies may be used for diagnosis. In addition, the data can also provide a full picture of noise interference that the IMD 101 encountered and allows new noise detection algorithm evaluation. The computation related to the diagnosis may be done at the IMD 101, cloud, or App 112 (e.g., smart phone, personal computer) level.

New EGM-based features for biomarker estimate can be developed. The continuously acquired EGMs may be acquired simultaneously with other sensor data such as glucose level, CMES pressure, heart sound, SpO2, activity, ketone, etc., to develop EGM based features to estimate the data the sensors 110, 234, 312 collect. For example, the EGM data may be compared against the continuous glucose level to predict EGM-based glucose monitoring for diabetic patients. Another example is the continuously acquired data that is collected during dialysis to compare the EGM changes with respect to the change in various blood biomarkers such as creatinine, troponin, BNP, etc.

Although FIG. 9 is described from the perspective of a workstation, such as workstation 1010 discussed further below, it should be understood that one or more steps may be accomplished separately or simultaneously at the IMD 101 level or by another appropriate device discussed herein.

At 902, one or more processors receive the data units from the IMD 101. In some embodiments, the one or more processors can also receive sensor data from the IMD 101 and/or from sensors 110, 234, 312. Health and testing data can also be received from other external devices and/or input by the practitioner. For example, the patient 106 may provide blood glucose levels they measured at certain test intervals. Other blood samples may be drawn at certain times and their results can be correlated temporally with the data units.

At 904, one or more processors determine whether any time gaps exist between consecutive data units. For example, data units 0-25 and 28-50 may be identified. A time gap of two data units exists between the data units 25 and 28. In some embodiments, the time gap is noted. In other embodiments, one or more processors can use AI and/or machine learning to interpolate, estimate, and the like to replace missing data.

At 906, one or more processors align the data units temporally to form a combined dataset.

At 908, one or more processors align the sensor data (if any) and other patient related data (if any) temporally with the data units in the dataset. In some embodiments, one or more processors form a combined dataset including the sensor and/or other identified data.

At 910, one or more processors analyze the dataset and/or combined dataset. In other embodiments, datasets and/or combined datasets acquired at different times and/or from different patients can be compared. The analysis can be a computer-implemented method that uses one or a combination of different models, techniques, statistical tools, AI algorithms, and/or machine learning. For example, at least one of the following can be used: artificial neural networks, Monte Carlo analysis techniques, a Bayesian network, a statistical-based anomaly detection technique, one or more Markov models, knowledge-based techniques, neural networks, clustering and outlier detection, demographic analysis, genetic algorithms, and/or fuzzy logic techniques.

Based on the analysis, at 912, one or more processors can identify relationships between the acquired data and the health of the patient 106, and identify relationships between the continuously acquired data, the marker data, and sensor data, etc.

At 914, one or more processors propose and/or deliver treatment to transform certain conditions within the patient 106. In some embodiments, certain physiological events or features may be identified that can be used to trigger a subsequent CDCM. For example, if an event occurred during a stress test, one or more processors can identify a physiological event in the data that can be monitored for. When the IMD 101 detects the physiological event, the CDCM can be enabled. This monitoring generates a treatment that is customized for the patient 106, resulting in better outcomes for the patient 106. In another embodiment, one or more processors can generate reports and recommendations based on an evaluation of the health of the patient, diagnose disease, diagnose worsening of disease, etc., and suggest medication, medication changes, surgery, medical procedures, new and/or modified CDCMs based on specific times and/or physiological triggers, and the like, based on the analysis. In accordance with new and unique aspects, the customized treatment for the patient 106 leads to better outcomes for the patient 106.

### Distributed System

FIG. 10 illustrates the distributed processing system 1000 in accordance with embodiments herein. The distributed processing system 1000 includes a server 1002 connected to a database 1004, a programmer 1006, at least one of a local RF transceiver, which can be at least one of an external device 108 of various embodiments, and a user workstation 1010 electrically connected to a communication system 1012. The one or more server 1002 and one or more database 1004 can comprise a continuous data collection system 1030 that stores patient data associated with CDCM. For example, the external device 108 may be a tablet computer 108a, a smartphone 108b, a laptop computer 108c, and the like. In some embodiments, the external device 108 is associated with the patient, and the patient is instructed to keep the external device 108 close to their person, the battery charged, and the App 112 (FIG. 1) associated with CDCMs open to facilitate enabling and disabling a CDCM and the transfer of continuously acquired data from the IMD 101 to the personal device 108.

The communication system 1012 may be the internet, a voice over IP (VOIP) gateway, a local plain old telephone service (POTS) such as a public switched telephone network (PSTN), a cellular phone based network, and the like. Alternatively, the communication system 1012 may be a local area network (LAN), a campus area network (CAN), a metropolitan area network (MAN), or a wide area network (WAM). The communication system 1012 serves to provide a network that facilitates the transfer/receipt of information such as cardiac signal waveforms, ventricular and atrial heart rates.

The server 1002 is a computer system that provides services to other computing systems over a computer network. The server 1002 controls the communication of information such as cardiac signal waveforms, ventricular and atrial heart rates, and detection thresholds. The server 1002 interfaces with the communication system 1012 to transfer information between the programmer 1006, the external device 108, the user workstation 1010 as well as a cell phone 1014 and a personal data assistant (PDA) 1016 to the database 1004 for storage/retrieval of records of information. On the other hand, the server 1002 may upload raw and/or processed signals from an implanted lead 1022, the physiological sensor 110, and/or the IMD 101 via the external device 108 or the programmer 1006.

The database 1004 stores information such as cardiac signal waveforms, ventricular and atrial heart rates, physiological data, sensor data, and the like, for a single or multiple patients. The information is downloaded into the database 1004 via the server 1002 or, alternatively, the information is uploaded to the server from the database 1004. The programmer 1006 is similar to the external device 226, 301 and may reside in a patient's home, a hospital, or a physician's office. The programmer 1006 interfaces with the lead 1022 and the IMD 101. The programmer 1006 may wirelessly communicate with the IMD 101 and utilize protocols, such as Bluetooth, Bluetooth Low Energy (BLE), GSM, infrared wireless LANS, HIPERLAN, 3G, satellite, as well as circuit and packet data protocols, and the like. Alternatively, a hard-wired connection may be used to connect the programmer 1006 to the IMD 101. The programmer 1006 is able to acquire cardiac signals from the surface of a person (e.g., ECGs), intra-cardiac electrogram (e.g., IEGM) signals from the IMD 101, and/or cardiac signal waveforms, ventricular and atrial heart rates, etc., from the IMD 101. The programmer 1006 interfaces with the communication system 1012, either via the internet or via POTS, to upload the information acquired from the lead 1022 or the IMD 101 to the server 1002.

The external device 108 interfaces with the communication system 1012 to upload one or more sets of continuously acquired data, such as a plurality of continuously acquired data units of EGM data and marker data to the server 1002. The external device 108 can also upload sets of data or other data associated with the physiological sensor 110. In one embodiment, the IMD 101 and physiological sensor 110 have bi-directional connections 1024 with the external device 108 via a wireless connection. In other embodiments, the IMD 101 and the physiological sensor 110 can have single or bi-direction wireless connections 1028. The external device 108 is able to acquire cardiac signals from the surface of a person, intra-cardiac electrogram signals from the IMD 101, and/or cardiac signal waveforms, marker data, ventricular and atrial heart rates, etc., from the IMD 101, and physiological data from the physiological sensor 110, such as blood glucose level. On the other hand, the external device 108 may download instructions, detection thresholds, CDCM(s), and the like, from the database 1004 to the IMD 101 and/or physiological sensor 110.

The user workstation 1010 may interface with the communication system 1012 via the internet or POTS to download cardiac signal waveforms, ventricular and atrial heart rates, EGM data, marker data, physiological data, and the like via the server 1002 from the database 1004. Alternatively, the user workstation 1010 may download raw data from the lead 1022 or IMD 101 via either the programmer 1006 or the external device 108. Once the user workstation 1010 has downloaded the cardiac signal waveforms, ventricular and atrial heart rates, or detection thresholds, the user workstation 1010 may process the information in accordance with one or more of the operations described above. The user workstation 1010 may download the information and notifications to the cell phone 1014, the PDA 1016, the external device 108, the programmer 1006, and/or to the server 1002 to be stored on the database 1004. For example, the user workstation 1010 may communicate data to the cell phone 1014 or PDA 1016 via a wireless communication link 1026.

As discussed previously herein, the external device 108 may upload the continuously acquired data and/or physiological data to the database 1004 of the distributed processing system 1000. The uploaded continuously acquired data may be available to be uploaded and/or downloaded by a physician from the database 1004 using one of the cell phone 1014, PDA 1016, workstation 1010, programmer 1006, external device 108, and the like.

The communication system 1012 can communicate to the external device 108 that the upload of the continuously acquired data to the database 1004 is complete. The App 112 can display notification on the external device 108 to the patient that the upload of data is complete.

At any of the level of the IMD 101, the external device 108, user workstation 1010, cell phone 1014, PDA 1016, programmer 1006, and/or server 1002, the data collected from the IMD 101 and one or more of the physiological sensor 110, 234, 312 can be integrated for comparison and analysis as discussed herein. In some embodiments, the IMD 101 transmits a first set of data to the external device at a first time and a second set of data at a second time that is subsequent to the first time, and wherein the second set of data was sensed subsequently with respect to the first set of data. Any of the devices as discussed herein can receive and combine the first and second sets of data temporally. In further embodiments, a treatment based on the combined dataset can be determined.

### External Device

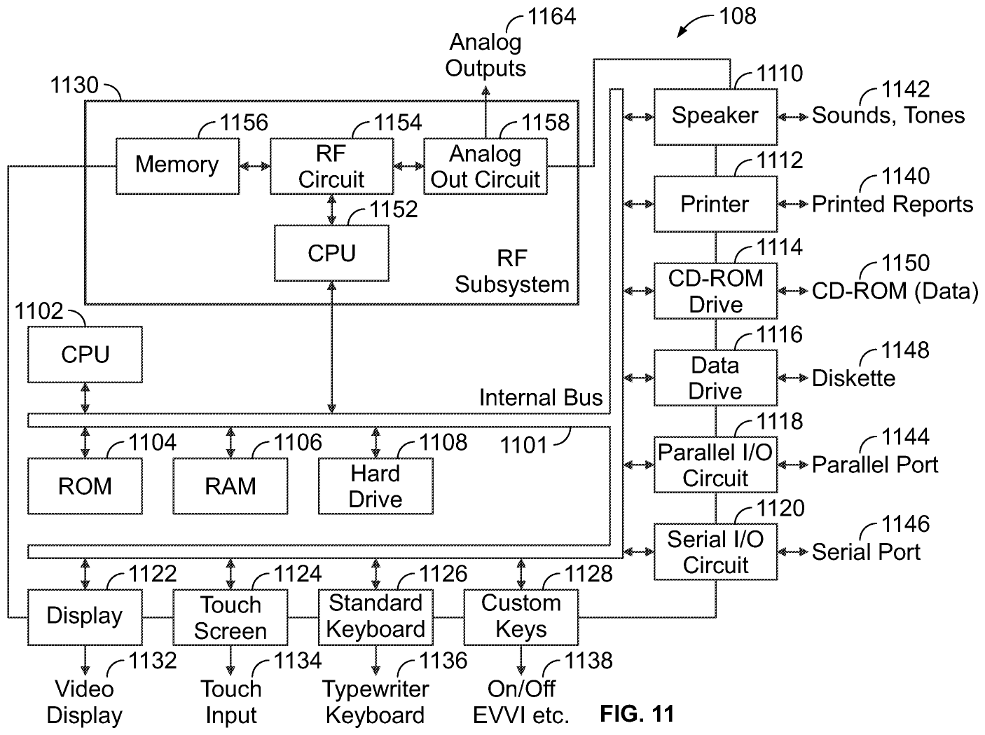

FIG. 11 illustrates a functional block diagram of the external device 108 that is operated in accordance with the processes described herein and to interface with the IMD 101 and/or physiological sensor 110 as described herein. The external device 108 may be an off-the-shelf device that performs the operations described herein from the instructions described above. Additionally or alternatively, the device 108 may be hard-wired with logic circuits to perform these operations. For example, the external device 108 may be a tablet computer, a smartphone, a laptop computer, a workstation, an IMD programmer, a PDA and/or the like located within a home of the patient 106, a hospital or clinic, an automobile, at an office of the patient, or the like. The external device 108 is for illustration purposes only, and it is understood that the circuitry could be duplicated, eliminated or disabled in any desired combination to provide a device capable of communicating with the IMD 101.

The external device 108 may include an internal bus 1101 that may connect/interface with a Central Processing Unit (“CPU”) 1102, ROM 1104, RAM 1106, a hard drive 1108, a speaker 1110, a printer 1112, a CD-ROM drive 1114, a floppy drive 1116, a parallel I/O circuit 1118, a serial I/O circuit 1120, a display 1122, a touchscreen 1124, a standard keyboard 1126, custom keys 1128, and an RF subsystem 1130. The internal bus 1101 is an address/data bus that transfers information between the various components described herein. The hard drive 1108 may store operational programs as well as data, such as stimulation waveform templates and detection thresholds.

The CPU 1102 typically includes a microprocessor, a micro-controller, or equivalent control circuitry, designed specifically to control interfacing with the external device 108 and with the IMD 101. The CPU 1102 may include RAM or ROM memory, logic and timing circuitry, state machine circuitry, and I/O circuitry to interface with the IMD 101. The display 1122 (e.g., may be connected to a video display 1132). The display 1122 displays various information related to the processes described herein. The touchscreen 1124 may display graphic information relating to the IMD 101 and include a graphical user interface. The graphical user interface may include graphical icons, scroll bars, buttons, and the like which may receive or detect user or touch inputs 1134 for the external device 108 when selections are made by the user. Optionally the touchscreen 1124 may be integrated with the display 1122. The keyboard 1126 (e.g., a typewriter keyboard 1136) allows the user to enter data to the displayed fields, as well as interface with the RF subsystem 1130. Furthermore, custom keys 1128 turn on/off 1138 (e.g., EVVI) the external device 108. The printer 1112 prints copies of reports 1140 for a physician to review or to be placed in a patient file, and the speaker 1110 provides an audible warning (e.g., sounds and tones 1142) to the user. The parallel I/O circuit 1118 interfaces with a parallel port 1044. The serial I/O circuit 1120 interfaces with a serial port 1146. The floppy drive 1116 accepts diskettes 1148. Optionally, the serial I/O port may be coupled to a USB port or other interface capable of communicating with a USB device such as a memory stick. The CD-ROM drive 1114 accepts CD ROMs 1150.

The RF subsystem 1130 includes a central processing unit (CPU) 1152 in electrical communication with an RF circuit 1154, which may communicate with both memory 1156 and an analog out circuit 1158. The memory 1156 may be configured to include a buffer memory or predetermined memory space that has a capacity for holding the transferred continuously acquired data. As discussed herein, the periodic communication interval can optionally be based on the capacity of the allotted memory space in the external device 108. The analog out circuit 1158 includes communication circuits to communicate with analog outputs 1164. The external device 108 may wirelessly communicate with the IMD 101 and utilize protocols, such as Bluetooth, BLE, ZigBee, MICS, and the like.

The microcontroller 210 (FIG. 2), 360 (of FIG. 3), the CPU 1102, and the CPU 1152 may execute a set of instructions that are stored in one or more storage elements, in order to process data. The storage elements may also store data or other information as desired or needed. The storage element may be in the form of an information source or a physical memory element within the microcontroller 210, 360, the CPU 1102, and the CPU 1152. The set of instructions may include various commands that instruct the microcontroller 360, the CPU 1102, and the CPU 1152 to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine. For example, the external device 108 may be equipped with at least one of an application software (App 112) that is available for patient interaction through a graphical icon on the touchscreen 1124. The application software may be programmed to initiate the external device 108 to request the communication link 104 between the external device 108 and the IMD 101 at the command of the patient 106.

### CLOSING

The various methods as illustrated in the Figures and described herein represent exemplary embodiments of methods. The methods may be implemented in software, hardware, or a combination thereof. In various of the methods, the order of the steps may be changed, and various elements may be added, reordered, combined, omitted, modified, etc. Various of the steps may be performed automatically (e.g., without being directly prompted by user input) and/or programmatically (e.g., according to program instructions).

Various modifications and changes may be made as would be obvious to a person skilled in the art having the benefit of this disclosure. It is intended to embrace all such modifications and changes and, accordingly, the above description is to be regarded in an illustrative rather than a restrictive sense.

Various embodiments of the present disclosure utilize at least one network that would be familiar to those skilled in the art for supporting communications using any of a variety of commercially-available protocols, such as Transmission Control Protocol/Internet Protocol (“TCP/IP”), User Datagram Protocol (“UDP”), protocols operating in various layers of the Open System Interconnection (“OSI”) model, File Transfer Protocol (“FTP”), Universal Plug and Play (“UpnP”), Network File System (“NFS”), Common Internet File System (“CIFS”) and AppleTalk. The network can be, for example, a local area network, a wide-area network, a virtual private network, the Internet, an intranet, an extranet, a public switched telephone network, an infrared network, a wireless network, a satellite network and any combination thereof.

In embodiments utilizing a web server, the web server can run any of a variety of server or mid-tier applications, including Hypertext Transfer Protocol (“HTTP”) servers, FTP servers, Common Gateway Interface (“CGI”) servers, data servers, Java servers, Apache servers and business application servers. The server(s) also may be capable of executing programs or scripts in response to requests from user devices, such as by executing one or more web applications that may be implemented as one or more scripts or programs written in any programming language, such as Java®, C, C# or C++, or any scripting language, such as Ruby, PHP, Perl, Python or TCL, as well as combinations thereof. The server(s) may also include database servers, including without limitation those commercially available from Oracle®, Microsoft®, Sybase® and IBM® as well as open-source servers such as MySQL, Postgres, SQLite, MongoDB, and any other server capable of storing, retrieving and accessing structured or unstructured data. Database servers may include table-based servers, document-based servers, unstructured servers, relational servers, non-relational servers or combinations of these and/or other database servers.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (“CPU” or “processor”), at least one input device (e.g., a mouse, keyboard, controller, touch screen or keypad) and at least one output device (e.g., a display device, printer or speaker). The terms “processor” and “computer,” and related terms, e.g., “processing device,” “computing device,” and “controller” may be not limited to just those integrated circuits referred to in the art as a computer, but refer to a microcontroller, a microcomputer, a programmable logic controller (PLC), field programmable gate array, and application specific integrated circuit, and other programmable circuits. Such a system may also include one or more storage devices, such as disk drives, optical storage devices and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device) and working memory as described above. The computer-readable storage media reader can be connected with, or configured to receive, a computer-readable storage medium, representing remote, local, fixed and/or removable storage devices as well as storage media for temporarily and/or more permanently containing, storing, transmitting and retrieving computer-readable information. The system and various devices also typically will include a number of software applications, modules, services or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Various embodiments may further include receiving, sending, or storing instructions and/or data implemented in accordance with the foregoing description upon a computer-readable medium. Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

All references, including publications, patent applications and patents, cited herein are hereby incorporated by reference to the same extent as if each reference were individually and specifically indicated to be incorporated by reference and were set forth in its entirety herein.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and physical characteristics described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

What is claimed is:

1. An implantable medical device (IMD), comprising:
one or more sensing circuits configured to sense one or more physiological characteristics and to generate physiological data indicative of the one or more physiological characteristics;
an input configured to receive a trigger;
a transceiver circuit configured to communicate with an external device;
a memory configured to store program instructions, the memory comprising a buffer memory; and
one or more processors, that when executing the program instructions, is configured to:
responsive to receiving the trigger:
enable a continuous data collection mode (CDCM) comprising a predetermined sampling rate;
continuously generate the physiological data;
continuously store the physiological data in the buffer memory at the predetermined sampling rate for a duration of a collection session associated with the CDCM, the amount of data stored in the buffer memory during the collection session, including the physiological data, exceeding a capacity of the buffer memory;
connect with the external device; and
transmit at least a portion of the physiological data stored in the buffer memory to the external device, wherein the connect and transmit operations are performed at a periodic communication interval during the collection session.

2. The IMD of claim 1, wherein the CDCM is further configured to store the physiological data at the predetermined sampling rate for a predetermined duration.

3. The IMD of claim 1, wherein the periodic communication interval is determined based on i) a capacity of the buffer memory, ii) a number of sensing channels configured to sense cardiac activity (CA) signals during the collection session, iii) the predetermined sampling rate, iv) a capacity of a memory space of the external device, v) data transfer speed between the IMD and the external device, or vi) time to establish connection between the IMD and the external device.

4. The IMD of claim 1, wherein the physiological data comprises i) heart sounds, ii) blood glucose data, iii) pulse oximetry, iv) CA signals, v) temperature, vi) heart rate, vii) impedance, viii) blood pressure, ix) blood oxygen saturation, x) activity, xi) posture, xii) nerve activity, xiii) blood sugar level, or xiv) cholesterol level.

5. The IMD of claim 1, wherein the trigger is i) a communication from the external device, ii) a physiological trigger from a physiological sensor located within the IMD or external to the IMD, or iii) generated in response to a physiological condition.

6. The IMD of claim 1, wherein the one or more processors is further configured to disable the CDCM based on i) a predetermined end time, ii) a predetermined duration, iii) a predetermined number of transmissions, or iv) receipt of a disabling message from a sensor or the external device.

7. The IMD of claim 1, wherein the one or more processors is further configured to determine a physiological feature, wherein in response to the physiological feature exceeding a threshold, the one or more processors are further configured to disable the CDCM.

8. The IMD of claim 7, wherein the physiological feature is a heart rate.

9. The IMD of claim 1, wherein, in response to receiving a disabling message, the one or more processors is further configured to disable the CDCM.

10. The IMD of claim 1, wherein the connect operation further comprises connecting with the external device at least a first time and a second time, wherein the transmit operation further comprises transmitting a first set of data during the first time and transmitting a second set of data during the second time that is different from the first set of data.

11. A computer implemented method, comprising:
responsive to receiving, by an implantable medical device (IMD), a trigger, enabling a continuous data collection mode (CDCM) on the IMD, the CDCM having an associated duration of a collection session;
continuously sensing physiological characteristics;
storing, at a predetermined sampling rate associated with the CDCM, physiological data associated with the sensed physiological characteristics in a buffer memory within the IMD, wherein an amount of data, including the physiological data, to be stored in the buffer memory during the collection session exceeding a capacity of the buffer memory; and
transmitting the data stored in the buffer memory from the IMD to an external device at a periodic communication interval set to prevent the data in the buffer memory from being overwritten during the collection session.

12. The method of claim 11, further comprising:
identifying one or more sensing channel associated with the CDCM, the one or more sensing channel included within the IMD;
wherein the continuously sensing further comprises continuously sensing physiological characteristics using the one or more sensing channel, wherein the physiological characteristic comprises cardiac activity (CA) signals; and
storing, at the predetermined sampling rate, the physiological data associated with the physiological characteristics sensed on the one or more sensing channel in the buffer memory.

13. The method of claim 11, wherein the transmitting further comprises:
transmitting a first set of data to the external device at a first time based on the periodic communication interval; and
transmitting a second set of data to the external device at a second time based on the periodic communication interval, wherein the second time is subsequent to the first time, wherein the second set of data was sensed subsequently with respect to the first set of data.

14. The method of claim 13, further comprising combining the first set of data and the second set of data temporally.

15. The method of claim 13, further comprising determining a treatment based on a combined dataset including the first set of data and the second set of data.

16. The method of claim 11, further comprising:
in response to enabling the CDCM, identifying marker data, wherein the marker data includes i) timing of QRS, ii) arrhythmia detection and termination, iii) timing of sensing, iv) noise, v) activity, vi) sleep, vii) physiological events, or viii) posture of patient, wherein the physiological data is EGM data; and
the storing further comprising storing the marker data with the EGM data, wherein the marker data is temporally associated with the EGM data.

17. The method of claim 11, wherein the periodic communication interval is determined based on i) a capacity of the buffer memory, ii) a number of sensing channels associated with the CDCM, or iii) the predetermined sampling rate.

18. The method of claim 11, further comprising:
determining a physiological feature; and
in response to the physiological feature exceeding a threshold, disabling the CDCM.

19. The method of claim 11, further comprising responsive to receiving, by the IMD, a second trigger, disabling the CDCM on the IMD.

20. The method of claim 19, further comprising responsive to the CDMC being disabled, transmitting the data stored in the buffer memory from the IMD to the external device.
